(19) United States
(12) Patent Application Publication
(10) Pub. No.: US 2010/0173777 A1
(43) Pub. Date: Jul. 8, 2010

(54) Piperazine Compounds Having Herbicidal Action

(75) Inventors: Eike Hupe, Mannheim (DE); Thomas Seitz, Viernheim (DE); Matthias Witschel, Bad Duerkheim (DE); Dschun Song, Mannheim (DE); William Karl Moberg, Hassloch (DE); Liliana Parra Rapado, Offenburg (DE); Frank Stelzer, Mannheim (DE); Andrea Vescovi, Mannheim (DE); Trevor William Newton, Neustadt (DE); Robert Reinhard, Limburgerhof (DE); Bernd Sievernich, Hassloch (DE); Klaus Grossmann, Neuhofen (DE); Thomas Ehrhardt, Speyer (DE); Michael Rack, Eppelheim (DE); Elmar Kibler, Hassloch (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/663,781

(22) PCT Filed: Jun. 11, 2008
(86) PCT No.: PCT/EP2008/057328

§ 371 Date: Dec. 9, 2009

(30) Foreign Application Priority Data
Jun. 12, 2007 (EP) 07110125.7

(51) Int. Cl. A01N 43/60; C07D 241/04; A01P 13/00

(52) U.S. Cl. 504/235; 544/383

(57) ABSTRACT

The present invention relates to piperazine compounds of the general formula I defined below and to their use as herbicides. Moreover, the invention relates to compositions for crop protection and to a method for controlling unwanted vegetation.

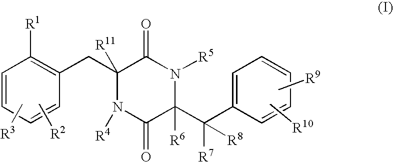

R¹ is selected from the group consisting of halogen, cyano, nitro, Z—C(═O)—R¹², phenyl and a 5- or 6-membered heterocyclic radical which has 1, 2, 3 or 4 heteroatoms selected from the group consisting of O, N and S as ring atoms, where phenyl and the heterocyclic radical are unsubstituted or may have 1, 2, 3 or 4 substituents R^{1a};
Z is a covalent bond or a CH₂ group;
R¹² is hydrogen, C₁-C₆-alkyl, C₃-C₆-cycloalkyl, C₂-C₆-alkenyl, C₅-C₆-cycloalkenyl, C₂-C₆-alkynyl and the like;
R² is hydrogen, halogen, nitro, cyano, C₁-C₄-alkyl, C₁-C₄-haloalkyl, C₂-C₄-alkenyl, C₁-C₄-alkoxy, C₁-C₄-haloalkoxy, benzyl or a group S(O)ₙR²¹ in which R²¹ is C₁-C₄-alkyl or C₁-C₄-haloalkyl and n is 0, 1 or 2;
R³ is hydrogen or halogen;
R⁴ is C₁-C₄-alkyl, C₃-C₄-alkenyl or C₃-C₄-alkynyl;
R⁵ is hydrogen, C₁-C₄-alkyl, C₃-C₄-alkenyl, C₃-C₄-alkynyl or a group C(═O)R⁵¹ in which R⁵¹ is hydrogen, C₁-C₄-alkyl, C₁-C₄-haloalkyl, C₁-C₄-alkoxy or C₁-C₄-haloalkoxy;
R⁶ is C₁-C₄-alkyl, C₁-C₄-hydroxyalkyl or C₁-C₄-haloalkyl;
R⁷, R⁸ independently of one another are hydrogen, OH, C₁-C₄-alkoxy, C₁-C₄-haloalkyloxy, C₁-C₄-alkyl or C₁-C₄-haloalkyl;
R⁹, R¹⁰ independently of one another are selected from the group consisting of hydrogen, halogen, CN, NO₂, C₁-C₄-alkyl, C₁-C₄-haloalkyl, C₂-C₄-alkenyl, C₁-C₄-alkoxy and C₁-C₄-haloalkoxy; and
R¹¹ is hydrogen or C₁-C₄-alkyl;
and the agriculturally useful salts of these compounds.

The present invention relates to piperazine compounds of the general formula I defined below and to their use as herbicides. Moreover, the invention relates to compositions for crop protection and to a method for controlling unwanted vegetation.

The thaxtomins A and B (King R. R. et al., J. Agric. Food Chem. (1992) 40, 834-837), which are produced by the plant pathogen S. scabies, are natural products having a central piperazine-2,5-dione ring which carries a 4-nitroindol-3-ylmethyl radical in the 3-position and an optionally OH-substituted benzyl radical in the 2-position. Because of their plant-damaging activity, this class of compounds was also examined for a possible use as herbicides (King R. R. et al., J. Agric. Food Chem. (2001) 49, 2298-2301).

EP-A 181152 and EP-A 243122 describe structurally similar piperazine compounds and their use as antagonists of the platelet activating factor.

WO 99/48889, WO 01/53290 and WO 2005/011699 describe 2,5-diketopiperazine compounds having in one of the 3- and 6-positions a 4-imidazolyl radical which is attached via a methylene or methyne group and in the other 3- or 6-position a benzyl or benzylidene radical. These compounds have antitumor activity.

US 2003/0171379 A1 describes the use of mactanamide, a fungistatic diketopiperazine of the formula A,

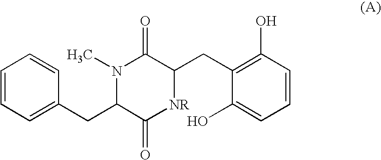

in which R is H or methyl, as antiinflammatory active compound in medicine.

The earlier patent application PCT/EP2006/070271 (=WO 2007/077201) describes 2,5-diketopiperazine compounds which have in each case an aryl or hetaryl radical attached via a methyne group in the 3-position and the 6-position.

It is an object of the present invention to provide compounds having herbicidal action. To be provided are in particular compounds which have high herbicidal activity, in particular even at low application rates, and which are sufficiently compatible with crop plants for commercial utilization.

These and further objects are achieved by the compounds of the formula I, defined below, and by their agriculturally suitable salts.

Accordingly, the present invention provides piperazine compounds of the general formula I

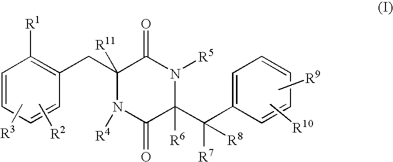

in which
R¹ is selected from the group consisting of halogen, cyano, nitro, Z—C(═O)—R¹², phenyl and a 5- or 6-membered heterocyclic radical which has 1, 2, 3 or 4 heteroatoms selected from the group consisting of O, N and S as ring atoms, where phenyl and the heterocyclic radical are unsubstituted or may have 1, 2, 3 or 4 substituents R^{1a} independently of one another selected from the group consisting of halogen, CN, NO₂, C₁-C₄-alkyl, C₁-C₄-alkoxy and C₁-C₄-haloalkoxy, and in which
Z is a covalent bond or a CH₂ group;
R¹² is hydrogen, C₁-C₆-alkyl, C₃-C₆-cycloalkyl, C₂-C₆-alkenyl, C₅-C₆-cycloalkenyl, C₂-C₆-alkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, C₁-C₆-alkylsulfonylamino, C₁-C₆-alkylaminosulfonylamino, [di-(C₁-C₆)-alkylamino]sulfonylamino, C₃-C₆-alkenylamino, C₃-C₆-alkynylamino, N—(C₂-C₆-alkenyl)-N—(C₁-C₆-alkyl)-amino, N—(C₂-C₆-alkynyl)-N—(C₁-C₆-alkyl)-amino, N—(C₁-C₆-alkoxy)-N—(C₁-C₆-alkyl)-amino, N—(C₂-C₆-alkenyl)-N—(C₁-C₆-alkoxy)-amino, N—(C₂-C₆-alkynyl)-N—(C₁-C₆-alkoxy)-amino, phenyl, phenoxy or phenylamino;
   where the alkyl moieties in the radicals listed under R¹² may be partially or fully halogenated and the phenyl moieties in the radicals listed under R¹² may carry 1, 2, 3 or 4 substituents R^{12a} selected from the group consisting of halogen, CN, NO₂, C₁-C₄-alkyl, C₁-C₄-haloalkyl, C₁-C₄-alkoxy and C₁-C₄-haloalkoxy;
R² is hydrogen, halogen, nitro, cyano, C₁-C₄-alkyl, C₁-C₄-haloalkyl, C₂-C₄-alkenyl, C₁-C₄-alkoxy, C₁-C₄-haloalkoxy, benzyl or a group S(O)ₙR²¹ in which R²¹ is C₁-C₄-alkyl or C₁-C₄-haloalkyl and n is 0, 1 or 2;
R³ is hydrogen or halogen;
R⁴ is C₁-C₄-alkyl, C₃-C₄-alkenyl or C₃-C₄-alkynyl;
R⁵ is hydrogen, C₁-C₄-alkyl, C₃-C₄-alkenyl, C₃-C₄-alkynyl or a group C(═O)R⁵¹ in which R⁵¹ is hydrogen, C₁-C₄-alkyl, C₁-C₄-haloalkyl, C₁-C₄-alkoxy or C₁-C₄-haloalkoxy;
R⁶ is C₁-C₄-alkyl, C₁-C₄-hydroxyalkyl or C₁-C₄-haloalkyl;
R⁷, R⁸ independently of one another are hydrogen, OH, C₁-C₄-alkoxy, C₁-C₄-haloalkyloxy, C₁-C₄-alkyl or C₁-C₄-haloalkyl;
R⁹, R¹⁰ independently of one another are selected from the group consisting of hydrogen, halogen, CN, NO₂, C₁-C₄-alkyl, C₁-C₄-haloalkyl, C₂-C₄-alkenyl, C₁-C₄-alkoxy and C₁-C₄-haloalkoxy; and
R¹¹ is hydrogen or C₁-C₄-alkyl;
and the agriculturally useful salts of these compounds.

The present invention also provides the use of piperazine compounds of the general formula I or the agriculturally useful salts of piperazine compounds of the formula I as herbicides, i.e. for controlling harmful plants.

The present invention also provides compositions comprising at least one piperazine compound of the formula I or an agriculturally useful salt of I and auxiliaries customary for formulating crop protection agents.

The present invention furthermore provides a method for controlling unwanted vegetation where a herbicidally effective amount of at least one piperazine compound of the formula I or an agriculturally useful salt of I is allowed to act on plants, their seeds and/or their habitat.

Moreover, the invention relates to processes and intermediates for preparing compounds of the formula I.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

With respect to the configuration of the two benzylic groups in the 3- and the 6-position, the compounds of the formula I may have the cis or the trans configuration. The invention provides both the pure cis isomers and trans isomers and their mixtures.

Both in the 3-position and in the 6-position of the piperazine ring, the compounds of the formula I have a center of chirality each. Accordingly, the compound I exists in the form of four different configurational isomers (S,S)-I, (R,R)-I, (R,S)-I and (S,R)-I where in each case two of these configurational isomers are like image and mirror image to one another, as shown in the figure below. Accordingly, the compound I may be present in the form of the pure enantiomers, and also as enantiomer mixtures, for example as a mixture of (S,S)-I with (R,R)-I or as a mixture of (R,S)-I with (S,R)-I, or as diastereomer mixtures, for example as a mixture of all four diastereomers.

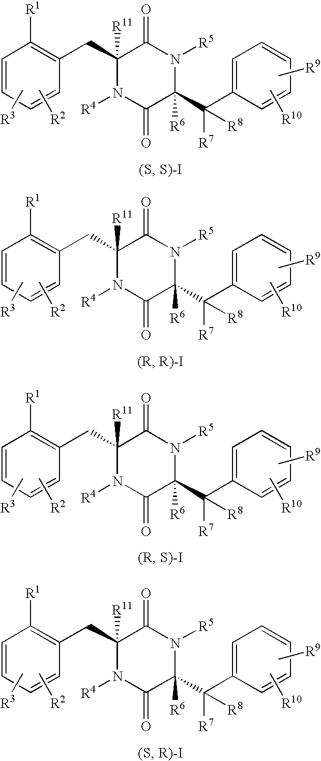

The invention provides both the pure enantiomers or diastereomers and their mixtures.

The compounds of the formula I may also be present in the form of their agriculturally useful salts, the nature of the salt generally being immaterial. Suitable salts are, in general, the salts of those cations or the acid addition salts of those acids whose cations and anions, respectively, which have no adverse effect on the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-yl-ammonium, di(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and the anions of C₁-C₄-alkanoic acids, preferably formate, acetate, propionate and butyrate.

The organic moieties mentioned for the substituents of the compounds according to the invention are collective terms for individual enumerations of the specific group members. All hydrocarbon chains, such as alkyl, haloalkyl, alkenyl, alkynyl, and also the alkyl moieties and alkenyl moieties in alkoxy, haloalkoxy, alkylamino, dialkylamino, N-alkylsulfonylamino, alkenyloxy, alkynyloxy, alkoxyamino, alkylaminosulfonylamino, dialkylaminosulfonylamino, alkenylamino, alkynylamino, N-(alkenyl)-N-(alkyl)-amino, N-(alkynyl)-N-(alkyl)-amino, N-(alkoxy)-N-(alkyl)-amino, N-(alkenyl)-N-(alkoxy)-amino or N-(alkynyl)-N-(alkoxy)-amino may be straight-chain or branched.

The prefix Cₙ-Cₘ— indicates the respective carbon number of the hydrocarbon moiety. Unless indicated otherwise, halogenated substituents preferably carry one to five identical or different halogen atoms, in particular fluorine atoms or chlorine atoms.

The term halogen denotes in each case fluorine, chlorine, bromine or iodine.

Examples of other meanings are:

alkyl and also the alkyl moieties, for example, in alkoxy, alkylamino, dialkylamino, N-alkylsulfonylamino, alkylaminosulfonylamino, dialkylaminosulfonylamino, N-(alkenyl)-N-(alkyl)-amino, N-(alkynyl)-N-(alkyl)-amino, N-(alkoxy)-N-(alkyl)-amino: saturated straight-chain or branched hydrocarbon radicals having one or more carbon atoms, for example 1 to 2, 1 to 4 or 1 to 6 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. In one embodiment according to the invention, alkyl denotes small alkyl groups such as C₁-C₄-alkyl. In another embodiment according to the invention, alkyl denotes relatively large alkyl groups such as C₅-C₆-alkyl.

Haloalkyl: an alkyl radical as mentioned above whose hydrogen atoms are partially or fully substituted by halogen atoms such as fluorine, chlorine, bromine and/or iodine, for example chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl.

Cycloalkyl and also the cycloalkyl moieties, for example, in cycloalkoxy or cycloalkylcarbonyl: monocyclic saturated hydrocarbon groups having three or more carbon atoms, for example 3 to 6 carbon ring members, such as cyclopropyl, cyclobutyl, cyclopentyl and cyclohexyl.

Alkenyl and also alkenyl moieties, for example in alkenylamino, alkenyloxy, N-(alkenyl)-N-(alkyl)-amino, N-(alkenyl)-N-(alkoxy)-amino: monounsaturated straight-chain or branched hydrocarbon radicals having two or more carbon atoms, for example 2 to 4, 2 to 6, or 3 to 6 carbon atoms, and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl.

Cycloalkenyl: monocyclic, monounsaturated hydrocarbon groups having from 5 to 6, preferably 5 to 6, carbon ring members, such as cyclopenten-1-yl, cyclopenten-3-yl, cyclohexen-1-yl, cyclohexen-3-yl, cyclohexen-4-yl.

Alkynyl and also alkynyl moieties, for example in alkynyloxy, alkynylamino, N-(alkynyl)-N-(alkyl)-amino or N-(alkynyl)-N-(alkoxy)-amino: straight-chain or branched hydrocarbon groups having two or more carbon atoms, for example 2 to 4, 2 to 6, or 3 to 6 carbon atoms, and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Alkoxy: alkyl, as defined above, which is attached via an oxygen atom: for example methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

Aryl: monocyclic or polycyclic aromatic hydrocarbon radicals having 6 to 14 carbon atoms, such as phenyl, naphthyl, anthracenyl or phenanthrenyl, preferably phenyl or naphthyl.

A 5- or 6-membered heterocyclic radical: a heterocyclic radical which has 5 or 6 ring atoms, 1, 2, 3 or 4 ring atoms being heteroatoms selected from the group consisting of O, S and N, where the heterocyclic radical is saturated, partially unsaturated or aromatic. Examples of heterocyclic radicals are:
5-membered saturated rings attached via carbon, such as tetrahydrofuran-2-yl, tetrahydrofuran-3-yl, tetrahydrothien-2-yl, tetrahydrothien-3-yl, tetrahydropyrrol-2-yl, tetrahydropyrrol-3-yl, tetrahydropyrazol-3-yl, tetrahydropyrazol-4-yl, tetrahydroisoxazol-3-yl, tetrahydroisoxazol-4-yl, tetrahydroisoxazol-5-yl, 1,2-oxathiolan-3-yl, 1,2-oxathiolan-4-yl, 1,2-oxathiolan-5-yl, tetrahydroisothiazol-3-yl, tetrahydroisothiazol-4-yl, tetrahydroisothiazol-5-yl, 1,2-dithiolan-3-yl, 1,2-dithiolan-4-yl, tetrahydroimidazol-2-yl, tetrahydroimidazol-4-yl, tetrahydrooxazol-2-yl, tetrahydrooxazol-4-yl, tetrahydrooxazol-5-yl, tetrahydrothiazol-2-yl, tetrahydrothiazol-4-yl, tetrahydrothiazol-5-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-dithiolan-2-yl, 1,3-dithiolan-4-yl, 1,3,2-dioxathiolan-4-yl;
6-membered saturated rings attached via carbon, such as:
tetrahydropyran-2-yl, tetrahydropyran-3-yl, tetrahydropyran-4-yl, piperidin-2-yl, piperidin-3-yl, piperidin-4-yl, tetrahydrothiopyran-2-yl, tetrahydrothiopyran-3-yl, tetrahydrothiopyran-4-yl, 1,3-dioxan-2-yl, 1,3-dioxan-4-yl, 1,3-dioxan-5-yl, 1,4-dioxan-2-yl, 1,3-dithian-2-yl, 1,3-dithian-4-yl, 1,3-dithian-5-yl, 1,4-dithian-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, 1,2-dithian-3-yl, 1,2-dithian-4-yl, hexahydropyrimidin-2-yl, hexahydropyrimidin-4-yl, hexahydropyrimidin-5-yl, hexahydropyrazin-2-yl, hexahydropyridazin-3-yl, hexahydropyridazin-4-yl, tetrahydro-1,3-oxazin-2-yl, tetrahydro-1,3-oxazin-4-yl, tetrahydro-1,3-oxazin-5-yl, tetrahydro-1,3-oxazin-6-yl, tetrahydro-1,3-thiazin-2-yl, tetrahydro-1,3-thiazin-4-yl, tetrahydro-1,3-thiazin-5-yl, tetrahydro-1,3-thiazin-6-yl, tetrahydro-1,4-thiazin-2-yl, tetrahydro-1,4-thiazin-3-yl, tetrahydro-1,4-oxazin-2-yl, tetrahydro-1,4-oxazin-3-yl, tetrahydro-1,2-oxazin-3-yl, tetrahydro-1,2-oxazin-4-yl, tetrahydro-1,2-oxazin-5-yl, tetrahydro-1,2-oxazin-6-yl;
5-membered saturated rings attached via nitrogen, such as:
tetrahydropyrrol-1-yl, tetrahydropyrazol-1-yl, tetrahydroisoxazol-2-yl, tetrahydroisothiazol-2-yl, tetrahydroimidazol-1-yl, tetrahydrooxazol-3-yl, tetrahydrothiazol-3-yl;
6-membered saturated rings attached via nitrogen, such as:
piperidin-1-yl, hexahydropyrimidin-1-yl, hexahydropyrazin-1-yl, hexahydropyridazin-1-yl, tetrahydro-1,3-oxazin-3-yl, tetrahydro-1,3-thiazin-3-yl, tetrahydro-1,4-thiazin-4-yl, tetrahydro-1,4-oxazin-4-yl, tetrahydro-1,2-oxazin-2-yl;
5-membered partially unsaturated rings attached via carbon, such as:
2,3-dihydrofuran-2-yl, 2,3-dihydrofuran-3-yl, 2,5-dihydrofuran-2-yl, 2,5-dihydrofuran-3-yl, 4,5-dihydrofuran-2-yl, 4,5-dihydrofuran-3-yl, 2,3-dihydrothien-2-yl, 2,3-dihydrothien-3-yl, 2,5-dihydrothien-2-yl, 2,5-dihydrothien-3-yl, 4,5-dihydrothien-2-yl, 4,5-dihydrothien-3-yl, 2,3-dihydro-1H-pyrrol-2-yl, 2,3-dihydro-1H-pyrrol-3-yl, 2,5-dihydro-1H-pyrrol-2-yl, 2,5-dihydro-1H-pyrrol-3-yl, 4,5-dihydro-1H-pyrrol-2-yl, 4,5-dihydro-1H-pyrrol-3-yl, 3,4-dihydro-2H-pyrrol-2-yl, 3,4-dihydro-2H-pyrrol-3-yl, 3,4-dihydro-5H-pyrrol-2-yl, 3,4-dihydro-5H-pyrrol-3-yl, 4,5-dihydro-1H-pyrazol-3-yl, 4,5-dihydro-1H-pyrazol-4-yl, 4,5-dihydro-1H-pyrazol-5-yl, 2,5-dihydro-1H-pyrazol-3-yl, 2,5-dihydro-1H-pyrazol-4-yl, 2,5-dihydro-1H-pyrazol-5-yl, 4,5-dihydroisoxazol-3-yl, 4,5-dihydroisoxazol-4-yl, 4,5-dihydroisoxazol-5-yl, 2,5-dihydroisoxazol-3-yl, 2,5-dihydroisoxazol-4-yl, 2,5-dihydroisoxazol-5-yl, 2,3-dihydroisoxazol-3-yl, 2,3-dihydroisoxazol-4-yl, 2,3-dihydroisoxazol-5-yl, 4,5-dihydroisothiazol-3-yl, 4,5-dihydroisothiazol-4-yl, 4,5-dihydroisothiazol-5-yl, 2,5-dihydroisothiazol-3-yl, 2,5-dihydroisothiazol-4-yl, 2,5-dihydroisothiazol-5-yl, 2,3-dihydroisothiazol-3-yl, 2,3-dihydroisothiazol-4-yl, 2,3-dihydroisothiazol-5-yl, Δ³-1,2-dithiol-3-yl, Δ³-1,2-dithiol-4-yl, Δ³-1,2-dithiol-5-yl, 4,5-dihydro-1H-imidazol-2-yl, 4,5-dihydro-1H-imidazol-4-yl, 4,5-dihydro-1H-imidazol-5-yl, 2,5-dihydro-1H-imidazol-2-yl, 2,5-dihydro-1H-imidazol-4-yl, 2,5-dihydro-1H-imidazol-5-yl, 2,3-dihydro-1H-imidazol-2-yl, 2,3-dihydro-1H-imidazol-4-yl, 4,5-dihydrooxazol-2-yl, 4,5-dihydrooxazol-4-yl, 4,5-dihydrooxazol-5-yl, 2,5-dihydrooxazol-2-yl, 2,5-dihydrooxazol-4-yl, 2,5-dihydrooxazol-5-yl, 2,3-dihydrooxazol-2-yl, 2,3-dihydrooxazol-4-yl, 2,3-dihydrooxazol-5-yl, 4,5-dihydrothiazol-2-yl, 4,5-dihydrothiazol-4-yl, 4,5-dihydrothiazol-5-yl, 2,5-dihydrothiazol-2-yl, 2,5-dihydrothiazol-4-yl, 2,5-dihydrothiazol-5-yl, 2,3-dihydrothiazol-2-yl, 2,3-dihydrothiazol-4-yl, 2,3-dihydrothiazol-5-yl, 1,3-dioxol-2-yl, 1,3-dioxol-4-yl, 1,3-dithiol-2-yl, 1,3-dithiol-4-yl, 1,3-oxathiol-2-yl, 1,3-oxathiol-4-yl, 1,3-oxathiol-5-yl;
6-membered partially unsaturated rings attached via carbon, such as:
2H-3,4-dihydropyran-6-yl, 2H-3,4-dihydropyran-5-yl, 2H-3,4-dihydropyran-4-yl, 2H-3,4-dihydropyran-3-yl, 2H-3,4-dihydropyran-2-yl, 2H-3,4-dihydropyran-6-yl, 2H-3,4-dihydrothiopyran-5-yl, 2H-3,4-dihydrothiopyran-4-yl, 2H-3,4-dihydropyran-3-yl, 2H-3,4-dihydropyran-2-yl, 1,2,3,4-tetrahydropyridin-6-yl, 1,2,3,4-tetrahydropyridin-5-yl, 1,2,3,4-tetrahydropyridin-4-yl, 1,2,3,4-tetrahydropyridin-3-yl, 1,2,3,4-tetrahydropyridin-2-yl, 2H-5,6-dihydropyran-2-yl, 2H-5,6-dihydropyran-3-yl, 2H-5,6-dihydropyran-4-yl, 2H-5,6-dihydropyran-5-yl, 2H-5,6-dihydropyran-6-yl, 2H-5,6-dihydrothiopyran-2-yl, 2H-5,6-dihydrothiopyran-3-yl, 2H-5,6-dihydrothiopyran-4-yl, 2H-5,6-dihydrothiopyran-5-yl, 2H-5,6-dihydrothiopyran-6-yl, 1,2,5,6-tetrahydropyridin-2-yl, 1,2,5,6-tetrahydropyridin-3-yl, 1,2,5,6-tetrahydropyridin-4-yl, 1,2,5,6-tetrahydropyridin-5-yl, 1,2,5,6-tetrahydropyridin-6-yl, 2,3,4,5-tetrahydropyridin-2-yl, 2,3,4,5-tetrahydropyridin-3-yl, 2,3,4,5-tetrahydropyridin-4-yl, 2,3,4,5-tetrahydropyridin-5-yl, 2,3,4,5-tetrahydropyridin-6-yl, 4H-pyran-2-yl, 4H-pyran-3-yl-, 4H-pyran-4-yl, 4H-thiopyran-2-yl, 4H-thiopyran-3-yl, 4H-thiopyran-4-yl, 1,4-dihydropyridin-2-yl, 1,4-dihydropyridin-3-yl, 1,4-dihydropyridin-4-yl, 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 3,4-dihydropyridin-2-yl, 3,4-dihydropyridin-3-yl, 3,4-dihydropyridin-4-yl, 3,4-dihydropyridin-5-yl, 3,4-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2H-5,6-dihydro-1,2-oxazin-3-yl, 2H-5,6-dihydro-1,2-oxazin-4-yl, 2H-5,6-dihydro-1,2-oxazin-5-yl, 2H-5,6-dihydro-1,2-oxazin-6-yl, 2H-5,6-dihydro-1,2-thiazin-3-yl, 2H-5,6-dihydro-1,2-thiazin-4-yl, 2H-5,6-dihydro-1,2-thiazin-5-yl, 2H-5,6-dihydro-1,2-thiazin-6-yl, 4H-5,6-dihydro-1,2-oxazin-3-yl, 4H-5,6-dihydro-1,2-oxazin-4-yl, 4H-5,6-dihydro-1,2-oxazin-5-yl, 4H-5,6-dihydro-1,2-oxazin-6-yl, 4H-5,6-dihydro-1,2-thiazin-3-yl, 4H-5,6-dihydro-1,2-thiazin-4-yl, 4H-5,6-dihydro-1,2-thiazin-5-yl, 4H-5,6-dihydro-1,2-thiazin-6-yl, 2H-3,6-dihydro-1,2-oxazin-3-yl, 2H-3,6-dihydro-1,2-oxazin-4-yl, 2H-3,6-dihydro-1,2-oxazin-5-yl, 2H-3,6-dihydro-1,2-oxazin-6-yl, 2H-3,6-dihydro-1,2-thiazin-3-yl, 2H-3,6-dihydro-1,2-thiazin-4-yl, 2H-3,6-dihydro-1,2-thiazin-5-yl, 2H-3,6-dihydro-1,2-thiazin-6-yl, 2H-3,4-dihydro-1,2-oxazin-3-yl, 2H-3,4-dihydro-1,2-oxazin-4-yl, 2H-3,4-dihydro-1,2-oxazin-5-yl, 2H-3,4-dihydro-1,2-oxazin-6-yl, 2H-3,4-dihydro-1,2-thiazin-3-yl, 2H-3,4-dihydro-1,2-thiazin-4-yl, 2H-3,4-dihydro-1,2-thiazin-5-yl, 2H-3,4-dihydro-1,2-thiazin-6-yl, 2,3,4,5-tetrahydropyridazin-3-yl, 2,3,4,5-tetrahydropyridazin-4-yl, 2,3,4,5-tetrahydropyridazin-5-yl, 2,3,4,5-tetrahydropyridazin-6-yl, 3,4,5,6-tetrahydropyridazin-3-yl, 3,4,5,6-tetrahydropyridazin-4-yl, 1,2,5,6-tetrahydropyridazin-3-yl, 1,2,5,6-tetrahydropyridazin-4-yl, 1,2,5,6-tetrahydropyridazin-5-yl, 1,2,5,6-tetrahydropyridazin-6-yl, 1,2,3,6-tetrahydropyridazin-3-yl, 1,2,3,6-tetrahydropyridazin-4-yl, 4H-5,6-dihydro-1,3-oxazin-2-yl, 4H-5,6-dihydro-1,3-oxazin-4-yl, 4H-5,6-dihydro-1,3-oxazin-5-yl, 4H-5,6-dihydro-1,3-oxazin-6-yl, 4H-5,6-dihydro-1,3-thiazin-2-yl, 4H-5,6-dihydro-1,3-thiazin-4-yl, 4H-5,6-dihydro-1,3-thiazin-5-yl, 4H-5,6-dihydro-1,3-thiazin-6-yl, 3,4,5-6-tetrahydropyrimidin-2-yl, 3,4,5,6-tetrahydropyrimidin-4-yl, 3,4,5,6-tetrahydropyrimidin-5-yl, 3,4,5,6-tetrahydropyrimidin-6-yl, 1,2,3,4-tetrahydropyrazin-2-yl, 1,2,3,4-tetrahydropyrazin-5-yl, 1,2,3,4-tetrahydropyrimidin-2-yl, 1,2,3,4-tetrahydropyrimidin-4-yl, 1,2,3,4-tetrahydropyrimidin-5-yl, 1,2,3,4-tetrahydropyrimidin-6-yl, 2,3-dihydro-1,4-thiazin-2-yl, 2,3-dihydro-1,4-thiazin-3-yl, 2,3-dihydro-1,4-thiazin-5-yl, 2,3-dihydro-1,4-thiazin-6-yl, 2H-1,2-oxazin-3-yl, 2H-1,2-oxazin-4-yl, 2H-1,2-oxazin-5-yl, 2H-1,2-oxazin-6-yl, 2H-1,2-thiazin-3-yl, 2H-1,2-thiazin-4-yl, 2H-1,2-thiazin-5-yl, 2H-1,2-thiazin-6-yl, 4H-1,2-oxazin-3-yl, 4H-1,2-oxazin-4-yl, 4H-1,2-oxazin-5-yl, 4H-1,2-oxazin-6-yl, 4H-1,2-thiazin-3-yl, 4H-1,2-thiazin-4-yl, 4H-1,2-thiazin-5-yl, 4H-1,2-thiazin-6-yl, 6H-1,2-oxazin-3-yl, 6H-1,2-oxazin-4-yl, 6H-1,2-oxazin-5-yl, 6H-1,2-oxazin-6-yl, 6H-1,2-thiazin-3-yl, 6H-1,2-thiazin-4-yl, 6H-1,2-thiazin-5-yl, 6H-1,2-thiazin-6-yl, 2H-1,3-oxazin-2-yl, 2H-1,3-oxazin-4-yl, 2H-1,3-oxazin-5-yl, 2H-1,3-oxazin-6-yl, 2H-1,3-thiazin-2-yl, 2H-1,3-thiazin-4-yl, 2H-1,3-thiazin-5-yl, 2H-1,3-thiazin-6-yl, 4H-1,3-oxazin-2-yl, 4H-1,3-oxazin-4-yl, 4H-1,3-oxazin-5-yl, 4H-1,3-oxazin-6-yl, 4H-1,3-thiazin-2-yl, 4H-1,3-thiazin-4-yl, 4H-1,3-thiazin-5-yl, 4H-1,3-thiazin-6-yl, 6H-1,3-oxazin-2-yl, 6H-1,3-oxazin-4-yl, 6H-1,3-oxazin-5-yl, 6H-1,3-oxazin-6-yl, 6H-1,3-thiazin-2-yl, 6H-1,3-oxazin-4-yl, 6H-1,3-oxazin-5-yl, 6H-1,3-thiazin-6-yl, 2H-1,4-oxazin-2-yl, 2H-1,4-oxazin-3-yl, 2H-1,4-oxazin-5-yl, 2H-1,4-oxazin-6-yl, 2H-1,4-thiazin-2-yl, 2H-1,4-thiazin-3-yl, 2H-1,4-thiazin-5-yl, 2H-1,4-thiazin-6-yl, 4H-1,4-oxazin-2-yl, 4H-1,4-oxazin-3-yl, 4H-1,4-thiazin-2-yl, 4H-1,4-thiazin-3-yl, 1,4-dihydropyridazin-3-yl, 1,4-dihydropyridazin-4-yl, 1,4-dihydropyridazin-5-yl, 1,4-dihydropyridazin-6-yl, 1,4-dihydropyrazin-2-yl, 1,2-dihydropyrazin-2-yl, 1,2-dihydropyrazin-3-yl, 1,2-dihydropyrazin-5-yl, 1,2-dihydropyrazin-6-yl, 1,4-dihydropyrimidin-2-yl, 1,4-dihydropyrimidin-4-yl, 1,4-dihydropyrimidin-5-yl, 1,4-dihydropyrimidin-6-yl, 3,4-dihydropyrimidin-2-yl, 3,4-dihydropyrimidin-4-yl, 3,4-dihydropyrimidin-5-yl or 3,4-dihydropyrimidin-6-yl;
5-membered partially unsaturated rings attached via nitrogen, such as:
2,3-dihydro-1H-pyrrol-1-yl, 2,5-dihydro-1H-pyrrol-1-yl, 4,5-dihydro-1H-pyrazol-1-yl, 2,5-dihydro-1H-pyrazol-1-yl, 2,3-dihydro-1H-pyrazol-1-yl, 2,5-dihydroisoxazol-2-yl, 2,3-dihydroisoxazol-2-yl, 2,5-dihydroisothiazol-2-yl, 2,3-dihydroisoxazol-2-yl, 4,5-dihydro-1H-imidazol-1-yl, 2,5-dihydro-1H-imidazol-1-yl, 2,3-dihydro-1H-imidazol-1-yl, 2,3-dihydrooxazol-3-yl, 2,3-dihydrothiazol-3-yl, 1,2,4-Δ ⁴-oxadiazolin-2-yl, 1,2,4-Δ ²-oxadiazolin-4-yl, 1,2,4-Δ ³-oxadiazolin-2-yl, 1,3,4-Δ ²-oxadiazolin-4-yl, 1,2,4-Δ ⁵-thiadiazolin-2-yl, 1,2,4-Δ ³-thiadiazolin-2-yl, 1,2,4-Δ ²-thiadiazolin-4-yl, 1,3,4-Δ ²-thiadiazolin-4-yl, 1,2,3-Δ ²-triazolin-1-yl, 1,2,4-Δ ²-triazolin-1-yl, 1,2,4-Δ ²-triazolin-4-yl, 1,2,4-Δ ³-triazolin-1-yl, 1,2,4-Δ ¹-triazolin-4-yl;
6-membered partially unsaturated rings attached via nitrogen, such as:
1,2,3,4-tetrahydropyridin-1-yl, 1,2,5,6-tetrahydropyridin-1-yl, 1,4-dihydropyridin-1-yl, 1,2-dihydropyridin-1-yl, 2H-5,6-dihydro-1,2-oxazin-2-yl, 2H-5,6-dihydro-1,2-thiazin-2-yl, 2H-3,6-dihydro-1,2-oxazin-2-yl, 2H-3,6-dihydro-1,2-thiazin-2-yl, 2H-3,4-dihydro-1,2-oxazin-2-yl, 2H-3,4-dihydro-1,2-thiazin-2-yl, 2,3,4,5-tetrahydropyridazin-2-yl, 1,2,5,6-tetrahydropyridazin-1-yl, 1,2,5,6-tetrahydropyridazin-2-yl, 1,2,3,6-tetrahydropyridazin-1-yl, 3,4,5,6-tetrahydropyrimidin-3-yl, 1,2,3,4-tetrahydropyrazin-1-yl, 1,2,3,4-tetrahydropyrimidin-1-yl, 1,2,3,4-tetrahydropyrimidin-3-yl, 2,3-dihydro-1,4-thiazin-4-yl, 2H-1,2-oxazin-2-yl, 2H-1,2-thiazin-2-yl, 4H-1,4-oxazin-4-yl, 4H-1,4-thiazin-4-yl, 1,4-dihydropyridazin-1-yl, 1,4-dihydropyrazin-1-yl, 1,2-dihydropyrazin-1-yl, 1,4-dihydropyrimidin-1-yl or 3,4-dihydropyrimidin-3-yl;
5-membered heteroaromatic rings attached via carbon, such as:
2-furyl, 3-furyl, 2-thienyl, 3-thienyl, pyrrol-2-yl, pyrrol-3-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-2-yl, imidazol-4-yl, oxazol-2-yl, oxazol-4-yl, oxazol-5-yl, thiazol-2-yl, thiazol-4-yl, thiazol-5-yl, 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl, 1,2,3-triazol-4-yl, 1,2,4-triazol-3-yl, [1H]-tetrazol-5-yl and [2H]-tetrazol-5-yl;
6-membered heteroaromatic rings attached via carbon, such as:
pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl, 1,2,4-triazin-5-yl and 1,2,4-triazin-6-yl;
5-membered heteroaromatic rings attached via nitrogen, such as:
pyrrol-1-yl, pyrazol-1-yl, imidazol-1-yl, 1,2,3-triazol-1-yl, 1,2,4-triazol-1-yl, [1H]-tetrazol-1-yl and [2H]-tetrazol-2-yl.

The heterocycles mentioned above may be substituted in the manner stated. Sulfur atoms in the heterocycles mentioned above may be oxidized to S═O or S(═O)₂.

Other meanings are:
alkenyloxy: alkenyl as mentioned above which is attached via an oxygen atom;
alkynyloxy: alkynyl as mentioned above which is attached via an oxygen atom;
alkylamino: a group NHR in which R is alkyl as defined above;
[dialkyl]amino: a group NR′R in which R and R′ are alkyl as defined above;
alkoxyamino: a group NH(OR) in which R is alkyl as defined above;
alkylsulfonylamino: a group NHS(O)₂R;
alkylaminosulfonylamino: a group NHS(O)₂NHR in which R is alkyl as defined above;
[dialkylamino]sulfonylamino: a group NHS(O)₂NR′R in which R and R′ are alkyl as defined above;
alkenylamino: a group NHR in which R is alkenyl as defined above;
alkynylamino: a group NHR in which R is alkynyl as defined above;
N-(alkenyl)-N-(alkyl)-amino: a group NR′R in which R is alkenyl and R′ is alkyl as defined above;
N-(alkynyl)-N-(alkyl)-amino: a group NR′R in which R is alkynyl and R′ is alkyl as defined above;
N-(alkoxy)-N-(alkyl)-amino: a group NR′R in which R is alkyl and R′ is alkoxy as defined above;
N-(alkenyl)-N-(alkoxy)-amino: a group NR′R in which R is alkenyl and R′ is alkoxy as defined above; and
N-(alkynyl)-N-(alkoxy)-amino: a group NR′R in which R is alkynyl and R′ is alkoxy as defined above.

In a particular embodiment, the variables of the compounds of the formula I have the meanings below, these meanings—both on their own and in combination with one another—being particular embodiments of the compounds of the formula I:

R¹ is in particular cyano, nitro or a 5- or 6-membered heteroaromatic radical as defined above which preferably has either 1, 2, 3 or 4 nitrogen atoms or 1 oxygen or 1 sulfur atom and, if appropriate, 1 or 2 nitrogen atoms as ring members and which is unsubstituted or may have 1 or 2 substituents selected from R^{1a}.

In a first preferred embodiment of the invention, R¹ is cyano or nitro.

In a further preferred embodiment of the invention, R¹ is a 5- or 6-membered heteroaromatic radical as defined above which preferably has either 1, 2, 3 or 4 nitrogen atoms or 1 oxygen or 1 sulfur atom and, if appropriate, 1 or 2 nitrogen atoms as ring members and which is unsubstituted or may have 1 or 2 substituents selected from R^{1a}. Examples of preferred heteroaromatic radicals are pyridazin-3-yl, pyridazin-4-yl, pyrimidin-2-yl, pyrimidin-4-yl, pyrimidin-5-yl, pyrazin-2-yl, 2-furyl, 3-furyl, 2-thienyl, 3-thienyl, pyrazol-1-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-1-yl, imidazol-2-yl, imidazol-4-yl, imidazol-5-yl, oxazol-2-yl, oxazol-4-yl, oxazol-5-yl, thiazol-2-yl, thiazol-4-yl and thiazol-5-yl, in particular heteroaromatic radicals attached via carbon, such as pyrazol-3-yl, imidazol-5-yl, oxazol-2-yl, thiazol-2-yl, thiazol-4-yl, thiazol-5-yl, pyridin-2-yl, pyridin-3-yl, pyridin-4-yl, pyrimidin-2-yl, pyrimidin-4-yl, pyrimidin-5-yl, pyridazin-4-yl, pyrazin-2-yl, [1H]-tetrazol-5-yl and [2H]-tetrazol-5-yl, where the heterocycles mentioned here in an exemplary manner may have 1 or 2 substituents selected from R^{1a}. Preferred radicals R^{1a} are in particular F, Cl, CN, nitro, methyl, ethyl, methoxy, ethoxy, difluoromethoxy, trifluoromethoxy and trifluoromethyl.

Preference is likewise given to compounds of the general formula I and salts thereof in which R¹ is halogen, in particular chlorine or bromine.

The radical R² is preferably hydrogen, fluorine, chlorine, C₁-C₂-alkyl, C₁-C₂-fluoroalkyl, ethenyl, C₁-C₂-alkoxy or C₁-C₂-fluoroalkoxy, in particular fluorine, chlorine, methyl, ethyl, methoxy, ethenyl or trifluoromethoxy. R² is especially preferred hydrogen, fluorine or chlorine.

From among the compounds of the formula I in which R² is different from hydrogen, preference is given to those compounds in which R² is located in the ortho-position to the point of attachment of the phenyl ring.

In a particularly preferred embodiment, R² is halogen, in particular chlorine or fluorine, which is located in the ortho-position to the point of attachment of the phenyl ring.

From among the compounds of the formula I in which R³ is halogen, preference is given to those compounds in which R³ is located in the para-position to the group R¹.

From among the compounds of the formula I in which R³ is halogen, preference is given to those compounds in which R³ is fluorine or chlorine. In another, likewise preferred, embodiment, R³ is hydrogen.

R⁴ is preferably methyl.

R⁵ is preferably hydrogen, methyl or ethyl, especially methyl.

Preference is likewise given to compounds of the formula I in which R⁵ is a group C(═O)R⁵¹ in which R⁵¹ has one of the meanings given above and is in particular hydrogen, C₁-C₄-alkyl, especially methyl or ethyl, or C₁-C₄-haloalkyl, especially C₁-C₂-fluoroalkyl, such as trifluoromethyl.

R⁶ is preferably C₁-C₃-alkyl, or C₁-C₂-fluoroalkyl, in particular methyl, ethyl, n-propyl, or trifluoromethyl, and especially methyl or ethyl.

Preferably at least one and in particular both radicals R⁷ and R⁸ are hydrogen.

From among the compounds of the formula I in which R⁹ is a radical different from hydrogen, preference is given to those compounds in which R⁹ is located in the para-position to the group CR⁷R⁸.

From among the compounds of the formula I in which R⁹ is a radical different from hydrogen, preference is given to those compounds in which R⁹ is halogen, in particular fluorine or chlorine. In another, likewise preferred, embodiment, R⁹ is hydrogen.

R¹⁰ is preferably hydrogen.

R¹¹ is preferably hydrogen.

In group C(O)R¹², R¹² is preferably hydrogen, C₁-C₄-alkyl or C₁-C₄-haloalkyl.

From among the compounds of the formula I and their salts, preference is given to the compounds of the general formula Ia and their agriculturally useful salts:

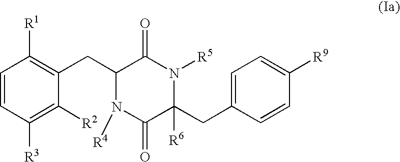

in which R¹, R², R³, R⁴, R⁵, R⁶ and R⁹ have one of the meanings given above, in particular the meanings given as being preferred. In formula Ia the radicals R¹, R², R³, R⁴, R⁵, R⁶ and R⁹ independently of one another, but preferably in combination, have in particular the meanings below:
R¹ cyano or nitro;
R² hydrogen, fluorine, chlorine, C₁-C₂-alkyl, ethenyl or C₁-C₂-alkoxy, in particular hydrogen, fluorine or chlorine;
R³ fluorine or hydrogen;
R⁴ methyl;
R⁵ hydrogen, methyl or ethyl, especially methyl;
R⁶ methyl or ethyl; and
R⁹ hydrogen or halogen, in particular hydrogen or fluorine.

As already explained above, the compounds of the formula I have a center of chirality each at the carbon atoms of the 3- and the 6-position of the piperazine ring. Preference is given to those compounds of the formula I in which the benzylic groups at the 3- and the 6-position have a cis configuration with respect to the piperazine ring, i.e. to the S,S enantiomer (S,S)-I and the R,R enantiomer (R,R)-I, and also their mixtures. Preference is likewise given to mixtures of the cis compound(s) with the trans compound(s), in which the cis compound(s) is/are present in excess, in particular to cis/trans mixtures having a cis/trans ratio of at least 2:1, in particular at least 5:1.

A particularly preferred embodiment of the invention relates to the S,S enantiomer of the formula (S,S)-I, and also to enantiomer mixtures and diastereomer mixtures of I in which the S,S enantiomer is the main component and is present in an amount of preferably at least 70%, in particular at least 80% and especially at least 90% of the compound I. Preference is likewise given to the agriculturally suitable salts of the enantiomers (S,S)-I and to enantiomer mixtures and diastereomer mixtures of the salts in which the S,S enantiomer is the main component and is present in an amount of preferably at least 70%, in particular at least 80% and especially at least 90% of the compound I. Another embodiment which is likewise preferred relates to a racemic mixture of the S,S enantiomer (S,S)-I with the R,R enantiomer (R,R)-I.

Another embodiment of the invention relates to the R,R enantiomer of the formula (R,R)-I, and also to enantiomer mixtures and diastereomer mixtures of I in which the R,R enantiomer is the main component and is present in an amount of preferably at least 70%, in particular at least 80% and especially at least 90% of the compound I. Another embodiment of the invention relates to the agriculturally suitable salts of the enantiomers (R,R)-I and to enantiomer mixtures and diastereomer mixtures of the salts in which the R,R enantiomer is the main component and is present in an amount of preferably at least 70%, in particular at least 80% and especially at least 90% of the compound I.

Preference is given in particular to the pure enantiomers of the formula (S,S)-Ia indicated below in which R¹, R², R³, R⁴, R⁵, R⁶ and R⁹ have one of the meanings given above, in particular one of the meanings given as being preferred or as being particularly preferred, and also to enantiomer mixtures and diastereomer mixtures of Ia in which the S,S enantiomer is the main component and is present in an amount of preferably at least 70%, in particular at least 80% and especially at least 90% of the compound Ia. Preference is likewise given to the agriculturally suitable salts of the enantiomers (S,S)-Ia and to enantiomer mixtures and diastereomer mixtures of the salts in which the salt of the S,S enantiomer is the main component and is present in an amount of preferably at least 70%, in particular at least 80% and especially at least 90% of the salt of Ia. Another embodiment which is likewise preferred relates to a racemic mixture of the S,S enantiomer (S,S)-Ia with the R,R enantiomer (R,R)-Ia.

Another embodiment of the invention relates to the pure enantiomers of the formula (R,R)-Ia indicated below in which R¹, R², R³, R⁴, R⁵, R⁶ and R⁹ have one of the meanings given above, in particular one of the meanings given as being preferred or as being particularly preferred, and also to enantiomer mixtures and diastereomer mixtures of Ia in which the R,R enantiomer is the main component and is present in an amount of preferably at least 70%, in particular at least 80% and especially at least 90% of the compound Ia. Another embodiment of the invention relates to the agriculturally suitable salts of the enantiomers (R,R)-Ia and to enantiomer mixtures and diastereomer mixtures of the salts in which the salt of the S,S enantiomer is the main component and is present in an amount of preferably at least 70%, in particular at least 80% and especially at least 90% of the salt of Ia.

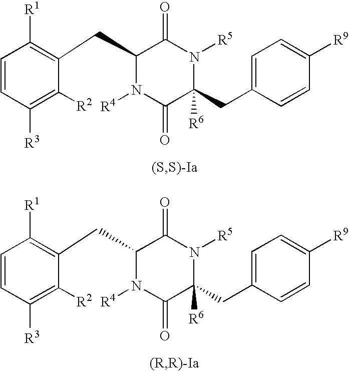

In formula (S,S)-Ia or (R,R)-Ia, the radicals R¹, R², R³, R⁴, R⁵, R⁶ and R⁹ independently of one another, but preferably in combination, have in particular the meanings below:
R¹ cyano or nitro;
R² hydrogen, fluorine, chlorine, C₁-C₂-alkyl, ethenyl or C₁-C₂-alkoxy, in particular hydrogen, fluorine or chlorine;
R³ fluorine or hydrogen;
R⁴ methyl;
R⁵ hydrogen, methyl or ethyl, especially methyl;
R⁶ methyl or ethyl; and
R⁹ hydrogen or halogen, in particular hydrogen or fluorine.

Examples of compounds which are preferred according to the invention are the compounds mentioned below and their salts:
2-[5-benzyl-1,4,5-trimethyl-3,6-dioxopiperazin-2-ylmethyl]benzonitrile,
2-[5-benzyl-1,4,5-trimethyl-3,6-dioxopiperazin-2-ylmethyl]-3-fluorobenzonitrile,
2-[5-benzyl-1,4,5-trimethyl-3,6-dioxopiperazin-2-ylmethyl]-3-methoxybenzonitrile,
2-[5-benzyl-1,4,5-trimethyl-3,6-dioxopiperazin-2-ylmethyl]-3,4-difluorobenzonitrile,
2-[5-benzyl-1,4,5-trimethyl-3,6-dioxopiperazin-2-ylmethyl]-3-methylbenzonitrile,
2-[5-benzyl-1,4,5-trimethyl-3,6-dioxopiperazin-2-ylmethyl]-3-ethenylbenzonitrile,
2-[5-benzyl-1,5-dimethyl-3,6-dioxopiperazin-2-ylmethyl]benzonitrile,
2-[5-benzyl-1,5-dimethyl-3,6-dioxopiperazin-2-ylmethyl]-3-fluorobenzonitrile,
2-[5-benzyl-1,5-dimethyl-3,6-dioxopiperazin-2-ylmethyl]-3-methoxybenzonitrile,
2-[5-benzyl-1,5-dimethyl-3,6-dioxopiperazin-2-ylmethyl]-3,4-difluorobenzonitrile,
2-[5-benzyl-1,5-dimethyl-3,6-dioxopiperazin-2-ylmethyl]-3-methylbenzonitrile,
2-[5-benzyl-1,5-dimethyl-3,6-dioxopiperazin-2-ylmethyl]-3-ethenylbenzonitrile,
2-[5-benzyl-5-ethyl-1,4-dimethyl-3,6-dioxopiperazin-2-ylmethyl]benzonitrile,
2-[5-benzyl-5-ethyl-1,4-dimethyl-3,6-dioxopiperazin-2-ylmethyl]-3-fluorobenzonitrile,
2-[5-benzyl-5-ethyl-1,4-dimethyl-3,6-dioxopiperazin-2-ylmethyl]-3-methoxybenzonitrile,
2-[5-benzyl-5-ethyl-1,4-dimethyl-3,6-dioxopiperazin-2-ylmethyl]-3,4-difluorobenzonitrile,
2-[5-benzyl-5-ethyl-1,4-dimethyl-3,6-dioxopiperazin-2-ylmethyl]-3-methylbenzonitrile,
2-[5-benzyl-5-ethyl-1,4-dimethyl-3,6-dioxopiperazin-2-ylmethyl]-3-ethenylbenzonitrile,
2-[5-benzyl-5-ethyl-1-methyl-3,6-dioxopiperazin-2-ylmethyl]benzonitrile,
2-[5-benzyl-5-ethyl-1-methyl-3,6-dioxopiperazin-2-ylmethyl]-3-fluorobenzonitrile,
2-[5-benzyl-5-ethyl-1-methyl-3,6-dioxopiperazin-2-ylmethyl]-3-methoxybenzonitrile,
2-[5-benzyl-5-ethyl-1-methyl-3,6-dioxopiperazin-2-ylmethyl]-3,4-difluorobenzonitrile,
2-[5-benzyl-5-ethyl-1-methyl-3,6-dioxopiperazin-2-ylmethyl]-3-methylbenzonitrile,
2-[5-benzyl-5-ethyl-1-methyl-3,6-dioxopiperazin-2-ylmethyl]-3-ethenylbenzonitrile,
2-[5-(4-fluorobenzyl)-1,4,5-trimethyl-3,6-dioxopiperazin-2-ylmethyl]benzonitrile,
2-[5-(4-fluorobenzyl)-1,4,5-trimethyl-3,6-dioxopiperazin-2-ylmethyl]-3-fluorobenzonitrile,
2-[5-(4-fluorobenzyl)-1,4,5-trimethyl-3,6-dioxopiperazin-2-ylmethyl]-3-methoxybenzonitrile,
2-[5-(4-fluorobenzyl)-1,4,5-trimethyl-3,6-dioxopiperazin-2-ylmethyl]-3,4-difluorobenzonitrile,
2-[5-(4-fluorobenzyl)-1,4,5-trimethyl-3,6-dioxopiperazin-2-ylmethyl]-3-methylbenzonitrile,
2-[5-(4-fluorobenzyl)-1,4,5-trimethyl-3,6-dioxopiperazin-2-ylmethyl]-3-ethenylbenzonitrile,
2-[5-(4-fluorobenzyl)-1,5-dimethyl-3,6-dioxopiperazin-2-ylmethyl]benzonitrile,
2-[5-(4-fluorobenzyl)-1,5-dimethyl-3,6-dioxopiperazin-2-ylmethyl]-3-fluorobenzonitrile,
2-[5-(4-fluorobenzyl)-1,5-dimethyl-3,6-dioxopiperazin-2-ylmethyl]-3-methoxybenzonitrile,
2-[5-(4-fluorobenzyl)-1,5-dimethyl-3,6-dioxopiperazin-2-ylmethyl]-3,4-difluorobenzonitrile,
2-[5-(4-fluorobenzyl)-1,5-dimethyl-3,6-dioxopiperazin-2-ylmethyl]-3-methylbenzonitrile,
2-[5-(4-fluorobenzyl)-1,5-dimethyl-3,6-dioxopiperazin-2-ylmethyl]-3-ethenylbenzonitrile,
2-[5-(4-fluorobenzyl)-5-ethyl-1,4-dimethyl-3,6-dioxopiperazin-2-ylmethyl]benzonitrile,
2-[5-(4-fluorobenzyl)-5-ethyl-1,4-dimethyl-3,6-dioxopiperazin-2-ylmethyl]-3-fluorobenzonitrile,
2-[5-(4-fluorobenzyl)-5-ethyl-1,4-dimethyl-3,6-dioxopiperazin-2-ylmethyl]-3-methoxybenzonitrile,
2-[5-(4-fluorobenzyl)-5-ethyl-1,4-dimethyl-3,6-dioxopiperazin-2-ylmethyl]-3,4-difluorobenzonitrile,
2-[5-(4-fluorobenzyl)-5-ethyl-1,4-dimethyl-3,6-dioxopiperazin-2-ylmethyl]-3-methylbenzonitrile,
2-[5-(4-fluorobenzyl)-5-ethyl-1,4-dimethyl-3,6-dioxopiperazin-2-ylmethyl]-3-ethenylbenzonitrile,
2-[5-(4-fluorobenzyl)-5-ethyl-1-methyl-3,6-dioxopiperazin-2-ylmethyl]benzonitrile,
2-[5-(4-fluorobenzyl)-5-ethyl-1-methyl-3,6-dioxopiperazin-2-ylmethyl]-3-fluorobenzonitrile,
2-[5-(4-fluorobenzyl)-5-ethyl-1-methyl-3,6-dioxopiperazin-2-ylmethyl]-3-methoxybenzonitrile,
2-[5-(4-fluorobenzyl)-5-ethyl-1-methyl-3,6-dioxopiperazin-2-ylmethyl]-3,4-difluorobenzonitrile,
2-[5-(4-fluorobenzyl)-5-ethyl-1-methyl-3,6-dioxopiperazin-2-ylmethyl]-3-methylbenzonitrile,
2-[5-(4-fluorobenzyl)-5-ethyl-1-methyl-3,6-dioxopiperazin-2-ylmethyl]-3-ethenylbenzonitrile,
3-benzyl-6-(2-nitrobenzyl)-1,3,4-trimethylpiperazine-2,5-dione,
3-benzyl-6-(2-fluoro-6-nitrobenzyl)-1,3,4-trimethylpiperazine-2,5-dione,
3-benzyl-6-(2,3-difluoro-6-nitrobenzyl)-1,3,4-trimethylpiperazine-2,5-dione,
3-benzyl-6-(2-methoxy-6-nitrobenzyl)-1,3,4-trimethylpiperazine-2,5-dione,
3-benzyl-6-(2-methyl-6-nitrobenzyl)-1,3,4-trimethylpiperazine-2,5-dione,
3-benzyl-6-(2-ethenyl-6-nitrobenzyl)-1,3,4-trimethylpiperazine-2,5-dione,
3-benzyl-6-(2-nitrobenzyl)-1,3-dimethylpiperazine-2,5-dione,
3-benzyl-6-(2-fluoro-6-nitrobenzyl)-1,3-dimethylpiperazine-2,5-dione,
3-benzyl-6-(2,3-difluoro-6-nitrobenzyl)-1,3-dimethylpiperazine-2,5-dione,
3-benzyl-6-(2-methoxy-6-nitrobenzyl)-1,3-dimethylpiperazine-2,5-dione,
3-benzyl-6-(2-methyl-6-nitrobenzyl)-1,3-dimethylpiperazine-2,5-dione,
3-benzyl-6-(2-ethenyl-6-nitrobenzyl)-1,3-dimethylpiperazine-2,5-dione,
3-benzyl-6-(2-nitrobenzyl)-3-ethyl-1,4-dimethylpiperazine-2,5-dione,
3-benzyl-6-(2-fluoro-6-nitrobenzyl)-3-ethyl-1,4-dimethylpiperazine-2,5-dione,
3-benzyl-6-(2,3-difluoro-6-nitrobenzyl)-3-ethyl-1,4-dimethylpiperazine-2,5-dione,
3-benzyl-6-(2-methoxy-6-nitrobenzyl)-3-ethyl-1,4-dimethylpiperazine-2,5-dione,
3-benzyl-6-(2-methyl-6-nitrobenzyl)-3-ethyl-1,4-dimethylpiperazine-2,5-dione,
3-benzyl-6-(2-ethenyl-6-nitrobenzyl)-3-ethyl-1,4-dimethylpiperazine-2,5-dione,
3-benzyl-6-(2-nitrobenzyl)-3-ethyl-1-methylpiperazine-2,5-dione,
3-benzyl-6-(2-fluoro-6-nitrobenzyl)-3-ethyl-1-methylpiperazine-2,5-dione,
3-benzyl-6-(2,3-difluoro-6-nitrobenzyl)-3-ethyl-1-methylpiperazine-2,5-dione,
3-benzyl-6-(2-methoxy-6-nitrobenzyl)-3-ethyl-1-methylpiperazine-2,5-dione,
3-benzyl-6-(2-methyl-6-nitrobenzyl)-3-ethyl-1-methylpiperazine-2,5-dione,
3-benzyl-6-(2-ethenyl-6-nitrobenzyl)-3-ethyl-1-methylpiperazine-2,5-dione,
3-(4-fluorobenzyl)-6-(2-nitrobenzyl)-1,3,4-trimethylpiperazine-2,5-dione,
3-(4-fluorobenzyl)-6-(2-fluoro-6-nitrobenzyl)-1,3,4-trimethylpiperazine-2,5-dione,
3-(4-fluorobenzyl)-6-(2,3-difluoro-6-nitrobenzyl)-1,3,4-trimethylpiperazine-2,5-dione,
3-(4-fluorobenzyl)-6-(2-methoxy-6-nitrobenzyl)-1,3,4-trimethylpiperazine-2,5-dione,
3-(4-fluorobenzyl)-6-(2-methyl-6-nitrobenzyl)-1,3,4-trimethylpiperazine-2,5-dione,
3-(4-fluorobenzyl)-6-(2-ethenyl-6-nitrobenzyl)-1,3,4-trimethylpiperazine-2,5-dione,
3-(4-fluorobenzyl)-6-(2-nitrobenzyl)-1,3-dimethylpiperazine-2,5-dione,
3-(4-fluorobenzyl)-6-(2-fluoro-6-nitrobenzyl)-1,3-dimethylpiperazine-2,5-dione,
3-(4-fluorobenzyl)-6-(2,3-difluoro-6-nitrobenzyl)-1,3-dimethylpiperazine-2,5-dione,
3-(4-fluorobenzyl)-6-(2-methoxy-6-nitrobenzyl)-1,3-dimethylpiperazine-2,5-dione,
3-(4-fluorobenzyl)-6-(2-methyl-6-nitrobenzyl)-1,3-dimethylpiperazine-2,5-dione,
3-(4-fluorobenzyl)-6-(2-ethenyl-6-nitrobenzyl)-1,3-dimethylpiperazine-2,5-dione,
3-(4-fluorobenzyl)-6-(2-nitrobenzyl)-3-ethyl-1,4-dimethylpiperazine-2,5-dione,
3-(4-fluorobenzyl)-6-(2-fluoro-6-nitrobenzyl)-3-ethyl-1,4-dimethylpiperazine-2,5-dione,
3-(4-fluorobenzyl)-6-(2,3-difluoro-6-nitrobenzyl)-3-ethyl-1,4-dimethylpiperazine-2,5-dione,
3-(4-fluorobenzyl)-6-(2-methoxy-6-nitrobenzyl)-3-ethyl-1,4-dimethylpiperazine-2,5-dione,
3-(4-fluorobenzyl)-6-(2-methyl-6-nitrobenzyl)-3-ethyl-1,4-dimethylpiperazine-2,5-dione,
3-(4-fluorobenzyl)-6-(2-ethenyl-6-nitrobenzyl)-3-ethyl-1,4-dimethylpiperazine-2,5-dione,
3-(4-fluorobenzyl)-6-(2-nitrobenzyl)-3-ethyl-1-methylpiperazine-2,5-dione,
3-(4-fluorobenzyl)-6-(2-fluoro-6-nitrobenzyl)-3-ethyl-1-methylpiperazine-2,5-dione,
3-(4-fluorobenzyl)-6-(2,3-difluoro-6-nitrobenzyl)-3-ethyl-1-methylpiperazine-2,5-dione,
3-(4-fluorobenzyl)-6-(2-methoxy-6-nitrobenzyl)-3-ethyl-1-methylpiperazine-2,5-dione,
3-(4-fluorobenzyl)-6-(2-methyl-6-nitrobenzyl)-3-ethyl-1-methylpiperazine-2,5-dione, and
3-(4-fluorobenzyl)-6-(2-ethenyl-6-nitrobenzyl)-3-ethyl-1-methylpiperazine-2,5-dione.

From among the compounds mentioned here in an exemplary manner and their salts, preference is given to those compounds and salts in which the benzylic groups in the 3- and the 6-position of the piperazine ring are in a cis configuration to one another, and to mixtures of the cis compound(s) with the trans compound(s) in which the cis compound(s) is/are present in excess, in particular to cis/trans mixtures having a cis/trans ratio of at least 2:1, in particular at least 5:1.

From among the compounds mentioned here in an exemplary manner and their salts, preference is in each case given to the S,S enantiomer and its salts. Preference is likewise given to enantiomer mixtures and diastereomer mixtures of the compounds I mentioned here in an exemplary manner and their salts in which the S,S enantiomer is the main component and is present in an amount of preferably at least 70%, in particular at least 80% and especially at least 90% of the compound in question. Another embodiment which is likewise preferred relates to racemic mixtures of the S,S enantiomer in question with the R,R enantiomer in question of the compounds listed here in an exemplary manner.

The compounds according to the invention can be prepared by standard processes of organic chemistry, for example a process (hereinbelow referred to as process A) which comprises the following steps:
i) provision of a compound of the general formula II

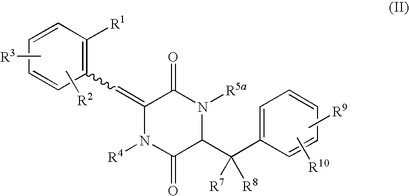

in which R¹, R², R³, R⁴, R⁷, R⁸, R⁹ and R¹⁵ have the meanings mentioned above, and R^{5a} has one of the meanings given for R⁵ different from hydrogen or is a protective group;
ii) reaction of the compound II with an alkylating agent of the formula R⁶—X in which R⁶ has the meanings given above and X is a nucleophilically displaceable leaving group, in the presence of a base; if appropriate removal of the protective group, if R^{5a} is a protective group;
   and
iii) hydrogenation of the compound obtained in step ii), which gives a compound of the formula I, in which R¹¹ is hydrogen;
   or
iia) hydrogenation of the compound II;
   and
iiia) reaction of the compound obtained in step iia) with an alkylating agent of the formula R⁶—X in which R⁶ has the meanings given above and X is a nucleophilically displaceable leaving group, in the presence of a base and if appropriate removal of the protective group, if R^{5a} is a protective group, which gives a compound of the formula I, in which R¹¹ is hydrogen;
iv) and, if appropriate, reaction of the compound of formula I, in which R¹¹ is hydrogen with an alkylating agent R¹¹—X in which R¹¹ is C₁-C₄-alkyl and X is a nucleophilically displaceable leaving group, in the presence of a base.

The alkylation in step ii) or iiia) can be carried out analogously to standard processes of alkylation, for example according to the methods described by I. O. Donkor et al., Bioorg. Med. Chem. Lett. 11 (19) (2001), 2647-2649, B. B. Snider et al., Tetrahedron 57 (16) (2001), 3301-3307, I. Yasuhiro et al., Heterocycles, 45, 1997, 1151, J. Am. Chem. Soc. 105, 1983, 3214, J. Am. Chem. Soc. 124(47) (2002), 14017-14019, Chem. Commun. 1998, 659 or M. Falorni et al., Europ. J. Org. Chem. (8) (2000), 1669-1675.

To this end, in the steps ii) the piperazine compound of the formula II is reacted with a suitable alkylating agent, hereinbelow compound X—R⁶, which gives a piperazine compound of the formula I (see, for example, J. Am. Chem. Soc. 105, 1983, 3214).

In the alkylating agents X—R⁶, X can be halogen, in particular chlorine, bromine or iodine, or O—SO₂—R^{m} with R^{m} having the meaning C₁-C₄-alkyl or aryl, which are optionally substituted by halogen, C₁-C₄-alkyl or halo-C₁-C₄-alkyl.

The reaction is usually carried out at temperatures in the range from −78° C. to the boiling point of the reaction mixture, preferably from −50° C. to 65° C., particularly preferably from −30° C. to 65° C. In general, the reaction is carried out in a solvent, preferably in an inert organic solvent.

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water, dimethyl sulfoxide, N-methylpyrrolidone, dimethylformamide and dimethylacetamide, and also morpholine and N-methylmorpholine and mixtures thereof. Preferred solvents are toluene, dichloromethane, tetrahydrofuran, N-methylpyrrolidone or dimethylformamide and mixtures thereof.

In general, the alkylation of the compound II in step ii) or iiia) is carried out using the alkylating agent in the presence of a base. Suitable bases are inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. The bases are generally employed in equimolar amounts. They can also be used in excess or even as solvent. In a preferred embodiment, the base is employed in an equimolar amount or an essentially equimolar amount. In a further preferred embodiment, the base used is sodium hydride.

Suitable protective groups as group R^{5a} are in particular the radicals C(O)R⁵¹ mentioned above, for example the acetyl radical. The introduction of these protective groups can be carried out analogously to known processes of protective group chemistry, for example by reaction with anhydrides of the formula (R⁵¹C(O))₂O, for example according to the method described in Green, Wuts, Protective Groups in Organic Synthesis, 3rd ed. 1999, John Wiley and Sons, p. 553.

The removal of a protective group R^{5a} can be carried out analogously to known processes of protective group chemistry (see Green, Wuts, Protective Groups in Organic Synthesis, 3rd ed. 1999, John Wiley and Sons, p. 553).

The hydrogenation in step iii) or in step Ha) can be carried out analogously to known processes for reducing C═C double bonds (see, for example, J. March, Advanced Organic Chemistry, 3rd ed. John Wiley and Sons 1985, pp. 690-700, and also Peptide Chemistry 17, 1980, pp. 59-64, Tetrahedron Lett. 46, 1979, pp. 4483-4486.

The hydrogenation is frequently carried out by reaction with hydrogen in the presence of transition metal catalysts, for example catalysts comprising Pt, Pd, Rh or Ru as active metal species. Suitable are both heterogeneous catalysts, such as supported Pd or Pt catalysts, for example Pd on activated carbon, furthermore PtO₂, and also homogeneous catalysts. By using stereoselective catalysts, it is possible to carry out an enantioselective hydrogenation of the double bond (see Peptide Chemistry 17, 1980, pp. 59-64, Tetrahedron Lett. 46, 1979, pp. 4483-4486).

The hydrogenation of II can be carried out either after the alkylation of II, i.e. in step iii), or prior to the alkylation of II, i.e. in step iia).

If appropriate, following the removal of the protective group in step ii) or iiia), which gives a compound I where R⁵═H, a new radical R⁵ different from hydrogen may be introduced by alkylation or by acylation. A subsequent alkylation or acylation for introducing the radical R⁵ can be carried out using standard processes of organic chemistry, for example the methods indicated above for steps ii) and iiia).

The preparation of the compound I in which R¹¹ is C₁-C₄-alkyl is expediently carried out by reacting the compound of the formula I in which R¹¹ is hydrogen with an alkylating agent R¹¹—X in which R¹¹ is C₁-C₄-alkyl and X is a nucleophilically displaceable leaving group, for example halogen, in particular chlorine, bromine or iodine, or O—SO₂—R^{m} where R^{m} has the meaning of C₁-C₄-alkyl or aryl which are optionally substituted by halogen, C₁-C₄-alkyl or halo-C₁-C₄-alkyl, in the presence of a base. With respect to the reaction conditions required for this reaction, what was said for the alkylation in step ii) or iiia) applies analogously.

Besides, compounds of the formula II are known, for example from PCT/EP2007/050067 (=WO 2007/077247), the entire content of which is hereby included be way of reference.

The compounds of the formula II can be prepared, for example, by reacting a benzaldehyde of the formula III with a piperazine compound IV in the context of an aldol condensation, as illustrated in the scheme below:

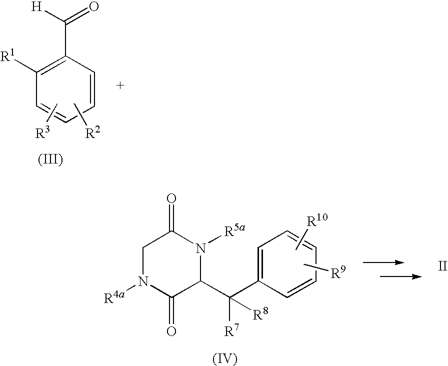

In the formulae III and IV, the variables R¹, R², R³, R^{5a}, R⁷, R⁸, R⁹ and R¹⁰ have the meanings given for formula II. R^{4a} is a protective group, C₁-C₄-alkyl, C₃-C₄-alkenyl or C₃-C₄-alkynyl. Suitable protective groups for the nitrogen atoms of the piperazine ring are in particular acyl groups, for example groups of the formula C(O)R⁵² in which R⁵² has one of the meanings given for R⁵¹ and is in particular C₁-C₄-alkyl, for example methyl.

Such aldol condensations can be carried out analogously to the processes described in J. Org. Chem. 2000, 65 (24), 8402-8405, Synlett 2006, 677 and J. Heterocycl. Chem. 1988, 25, 591, the entire contents of which are hereby included by way of reference.

The aldol condensation is typically carried out in the presence of suitable bases. Suitable bases are those which are usually employed for aldol condensations. Preference is given to using an alkali metal or alkaline earth metal carbonate as base, for example sodium carbonate, potassium carbonate or cesium carbonate or mixtures thereof.

The reaction is preferably carried out in an inert, preferably aprotic organic solvent. Examples of suitable solvents are in particular dichloromethane, dichloroethane, chlorbenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide, N-methylpyrrolidone and dimethylacetamide. Preferred solvents are in particular selected from the group consisting of dimethylformamide, N-methylpyrrolidone and dimethylacetamide.

The temperatures required for the aldol condensation are generally in the range of from 0° C. to the boiling point of the solvent used and in particular in the range of from 10 to 80° C.

For the reaction of III with IV, it has been found to be advantageous for the radicals R^{4a} and R^{5a} in the compound IV to represent an acyl group, for example a group of the formula R⁵²C(O)—, in which R⁵² has one of the meanings given for R⁵¹ and is in particular C₁-C₄-alkyl, for example methyl.

The introduction of these protective groups into the compound IV can be carried out analogously to known processes of protective group chemistry, for example by reacting the corresponding NH-free compound (the compound of the formula IV where R^{4a}, R^{5a}═H) with anhydrides of the formula (R⁵²C(O))₂O, for example according to the method described in Green, Wuts, Protective Groups in Organic Synthesis, 3rd ed. 1999, John Wiley and Sons, p. 553. The removal of a protective group R^{4a}, R^{5a} can be carried out analogously to known processes of protective group chemistry.

If the radicals R^{4a} and R^{5a} in the compound IV represent a protective group, for example an acyl group, these radicals are removed after the aldol condensation, which gives a compound of the formula II′,

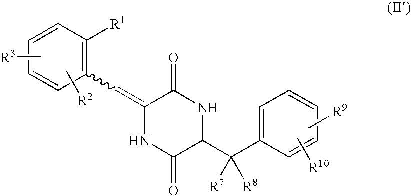

in which R¹, R², R³, R⁷, R⁸, R⁹ and R¹⁰ have the meanings above for formula I. The protective groups are generally removed by hydrolysis, the radical R^{4a} frequently already being cleaved off under the conditions of an aldol condensation. Into the resulting compound II′, the radical R⁴ is then introduced by alkylation and, if appropriate, the radical R⁵ is introduced by an alkylation or acylation.

The alkylation of the compound of the formula II′ for the introduction of the radicals R⁴ and R⁵ or R^{5a} can be carried out analogously to the methods given for step ii) and step iiia), for example according to the methods described in Heterocycles, 45, 1997, 1151, and Chem. Commun. 1998, 659.

To this end, the piperazine compound of the formula II′ is reacted with a suitable alkylating agent, hereinbelow compound X¹—R⁴ and X¹—R^{5a} or X¹—R^{5a}. In the alkylating agents X¹—R⁴, X¹—R⁵ and X¹—R^{5a}, X¹ may be halogen or O—SO₂—R^{m} where R^{m} has the meaning C₁-C₄-alkyl or aryl which are optionally substituted by halogen, C₁-C₄-alkyl or halo-C₁-C₄-alkyl. In the alkylating agents X¹—R⁴, X¹—R⁵ and X¹—R^{5a}, R⁴, R⁵ and R^{5a} independently of one another are C₁-C₄-alkyl, C₃-C₄-alkenyl or C₃-C₄-alkynyl.

The reaction of the compound II with the alkylating agent(s) X¹—R⁴, X¹—R⁵ and X¹—R^{5a} is preferably carried in the presence of a base. With respect to temperatures, bases and solvents, what was said for step ii) and iiia) applies in an analogous manner.

If R⁴ and R^{5a} in formula II or R⁴ and R⁵ in formula I are identical, the reaction of II′ with X¹—R⁴ and X¹—R⁵ or X¹—R^{5a} can be carried out simultaneously or successively in any order. If the radicals R⁴, R⁵ and R⁶ are identical, the reaction of II′ with X—R⁴ and X¹—R⁵ or X¹—R^{5a} can be carried out at the same time as step ii) of the process according to the invention.

If the radical R⁵ in formula I is an acyl group, this radical is introduced by an acylation before or after the hydrogenation of II. To this end, the compound I where R⁵═H is reacted with an acylating agent, hereinbelow compound X²—R⁵. In the acylating agent R⁵—X², R⁵ is a radical C(O)R⁵¹ in which R⁵¹ has the meanings mentioned above. X² is generally halogen, for example chlorine, or a group O—C(O)—R⁵¹. The reaction can be carried out analogously to the reaction of II with the alkylating agents X¹—R⁴ or X¹—R⁵.

The compound obtained in the alkylation of compound II in step ii) can also be prepared analogously to the preparation of II by reaction of the benzaldehyde compound III with a compound IVa:

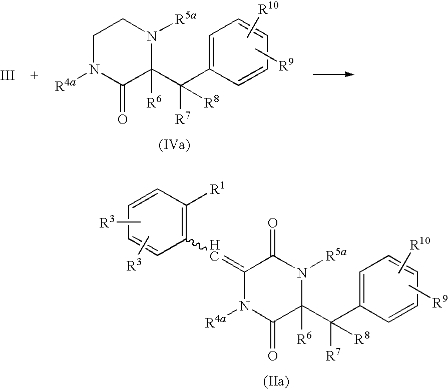

In this case, R¹, R², R³, R^{4a}, R^{5a}, R⁶, R⁷, R⁸, R⁹ and R¹⁰ have the meanings mentioned above, in particular one of the meanings mentioned as being preferred. Preferred radicals R^{4a} and R^{5a} in formula IVa are the acyl groups mentioned above of the formula R⁵²C(O) in which R⁵² has one of the meanings given for R⁵¹ and is in particular C₁-C₄-alkyl, for example methyl.

If R^{4a} and/or R^{5a} in formula IVa are protective groups/is a protective group, for example acyl group of the formula R⁵²C(O), the protective groups R^{4a} and/or R^{5a} will preferably be removed before the hydrogenation in step iii) of the process according to the invention. This gives a compound IIa in which R^{4a} and, if appropriate, R^{5a}, are hydrogen.

This compound IIa in which R^{4a} is hydrogen is reacted with an alkylating agent of the formula R⁴—X¹, preferably in the presence of a base before or after the hydrogenation. If R^{5a} is hydrogen, the compound IIa is reacted with, if appropriate, an alkylating agent of the formula R⁵—X¹ or an acylating agent R⁵—X², preferably in the presence of a base. For the reaction of compound IIa with the alkylating agents or acylating agents, what was said above for the reaction of II′ with the alkylating agent(s) or acylating agent(s) applies analogously.

The aldehyde III is either commercially available or can be synthesized according to known processes for preparing aldehydes.

The compounds of the formulae IV and IVa can be prepared by intramolecular cyclization of compounds of the general formula V and Va, respectively, analogously to other processes known from the literature, for example according to T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965.

If appropriate, the cyclization is followed by the introduction of a group R^{4a} or R^{5a} different from hydrogen if R^{4a} or R^{5b} in the formulae V and Va is hydrogen.

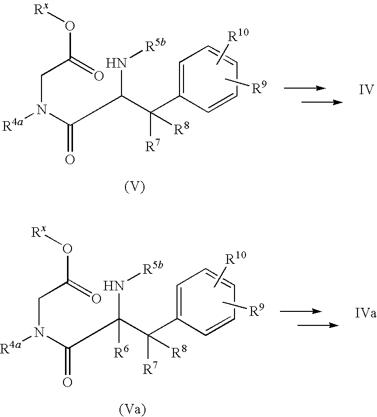

In the formulae V and Va, the variables R^{4a}, R⁶, R⁷, R⁸, R⁹ and R¹⁰ have the meanings mentioned above. R^{5b} is hydrogen, C₁-C₄-alkyl, C₃-C₄-alkenyl or C₃-C₄-alkynyl. R^{x} is here, for example, C₁-C₆-alkyl, in particular methyl or ethyl, or phenyl-C₁-C₆-alkyl, for example benzyl.

The cyclization of the compounds of the formula V or Va can be carried out in the presence of a base. In this case, the reaction is generally carried out at temperatures in the range of from 0° C. to the boiling point of the reaction mixture, preferably from 10° C. to 50° C., particularly preferably from 15° C. to 35° C. The reaction can be carried out in a solvent, preferably in an inert organic solvent.

Suitable inert organic solvents include aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water, and also dimethyl sulfoxide, dimethylformamide and dimethylacetamide, and also morpholine and N-methylmorpholine. It is also possible to use mixtures of the solvents mentioned. The preferred solvent is a tetrahydrofuran/water mixture having a mixing ratio of from 1:10 to 10:1.

Suitable bases are, for example, inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases. Preference is given in particular to potassium tert-butoxide, 2-hydroxypyridine or an aqueous solution of ammonia or a mixture of these bases. Preferably, only one of these bases is used. In a particularly preferred embodiment, the reaction is carried out in the presence of an aqueous solution of ammonia which may, for example, be of a strength of from 10 to 50% w/v. In another particularly preferred embodiment, the cyclization is carried out in a mixture comprising n-butanol or a mixture of butanol isomers (for example, a mixture of n-butanol and 2-butanol and/or isobutanol) and N-methylmorpholine, preferably under reflux conditions.

The cyclization of V or Va can also be carried out with acid catalysis, in the presence of activating compounds or thermally. The reaction of V in the presence of an acid is usually carried out at temperatures in the range of from 10° C. to the boiling point of the reaction mixture, preferably from 50° C. to the boiling point, particularly preferably at the boiling point under reflux. In general, the reaction is carried out in a solvent, preferably in an inert organic solvent.

Suitable solvents are, in principle, those which can also be used for the basic cyclization, in particular alcohols. In a preferred embodiment, the reaction is carried out in n-butanol or a mixture of different butanol isomers (for example a mixture of n-butanol and 2-butanol and/or isobutanol).

Suitable acids for the cyclization of V or Va are, in principle, both Brönstedt and Lewis acids. Use may be made in particular of inorganic acids, for example hydrohalic acids, such as hydrofluoric acid, hydrochloric acid, hydrobromic acid, inorganic oxoacids, such as sulfuric acid and perchloric acid, furthermore of inorganic Lewis acids, such as borin trifluoride, aluminum trichloride, iron(III) chloride, tin(IV) chloride, titanium(IV) chloride and zinc(II) chloride, and also of organic acids, for example carboxylic acids and hydroxycarboxylic acids, such as formic acid, acetic acid, propionic acid, oxalic acid, citric acid and trifluoroacetic acid, and also organic sulfonic acids, such as toluenesulfonic acid, benzenesulfonic acid, camphorsulfonic acid and the like. Of course, it is also possible to use a mixture of different acids.

In one embodiment of the process according to the invention, the reaction is carried out in the presence of organic acids, for example in the presence of carboxylic acids, such as formic acid, acetic acid or trifluoroacetic acid or a mixture of these acids. Preferably, only one of these acids is used. In a preferred embodiment, the reaction is carried out in acetic acid.

In a particularly preferred embodiment, the acidic cyclization is carried out in a mixture comprising n-butanol or a butanol isomer mixture (for example a mixture of n-butanol and 2-butanol and/or isobutanol), N-methylmorpholine and acetic acid, preferably under reflux conditions.

In a further embodiment of the invention, the conversion of V or Va is carried out by treatment with an activating agent in the presence of a base. In this case, R^{x} is hydrogen. An example of a suitable activating agent is di-(N-succinimidinyl) carbonate. Suitable activating agents are furthermore polystyrene- or non-polystyrene-supported di-cyclohexylcarbodiimide (DCC), diisopropylcarbodiimide, 1-ethyl-3-(dimethylaminopropyl)carbodiimide (EDAC), carbonyldiimidazole (CDI), chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCl) or sulfonyl chlorides, such as methanesulfonyl chloride, toluenesulfonyl chloride or benzenesulfonyl chloride. Suitable bases are the compounds cited for the basic cyclization. In one embodiment, the base used is triethylamine or N-ethyldiisopropylamine or mixtures thereof, particularly preferably N-ethyldiisopropylamine.

In a further embodiment of the invention, the conversion of V or Va is carried out exclusively by heating the reaction mixture (thermal cyclization). Here, the reaction is usually carried out at temperatures in the range of from 10° C. to the boiling point of the reaction mixture, preferably from 50° C. to the boiling point of the reaction mixture, particularly preferably at the boiling point of the reaction mixture under reflux. The reaction is generally carried out in a solvent, preferably in an inert organic solvent. In principle, suitable solvents are those solvents which can be used for the basic cyclization. Preference is given to polar aprotic solvents, for example dimethyl sulfoxide or dimethylformamide or mixtures thereof. In a preferred embodiment, the reaction is carried out in dimethyl sulfoxide.

If in compound V or Va one or both radicals R^{4a} and/or R^{5b} is/are hydrogen, the piperazine nitrogens can then, to introduce the radicals R^{4a} or R^{5a}, be alkylated using an alkylating agent R^{4a}—X¹ or R^{5a}—X¹ or be provided with a protective group by reaction with an acylating agent R^{4a}—X² or R^{5a}—X². Here, R^{4a}, R^{5a}, X¹ and X² have the meanings given above.

For their part, the compounds of the formula V or Va can be prepared by the scheme shown below analogously to processes from the literature, for example according to Wilford L. Mendelson et al., Int. J. Peptide & Protein Research 35(3), (1990), 249-57, Glenn L. Stahl et al., J. Org. Chem. 43(11), (1978), 2285-6 or A. K. Ghosh et al., Org. Lett. 3(4), (2001), 635-638.

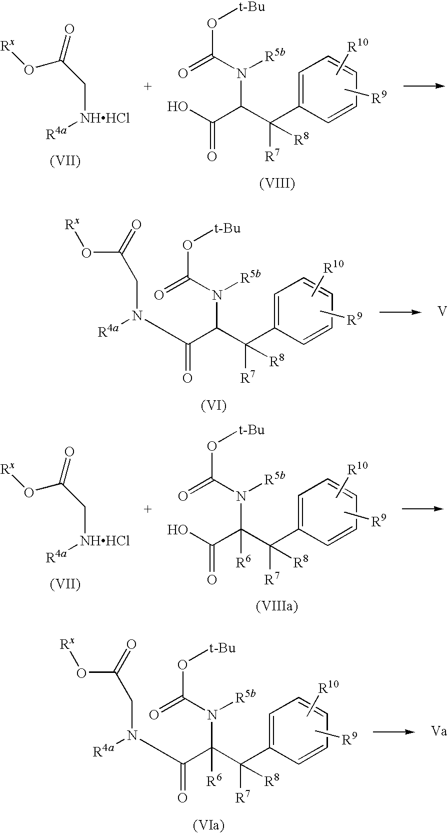

In the scheme, the variables R^{x}, R^{4a}, R^{5b}, R⁶, R⁷, R⁸, R⁹ and R¹⁰ have the meanings given for formula V. The synthesis comprises, in a first step, the coupling of glycine ester compounds of the formula VII with Boc-protected phenylalanine compounds VIII or VIIIa in the presence of an activating agent. Instead of Boc, it is also possible to use another amino-protective group.

The reaction of a compound of the formula VII with a compound of the formula VIII or VIIIa is usually carried out at temperatures in the range of from −30° C. to the boiling point of the reaction mixture, preferably of from 0° C. to 50° C., particularly preferably of from 20° C. to 35° C. The reaction can be carried out in a solvent, preferably in an inert organic solvent.

In general, the reaction requires the presence of an activating agent. Suitable activating agents are condensing agents, such as, for example, polystyrene- or non-polystyrene-supported dicyclohexylcarbodiimide (DCC), diisopropylcarbodiimide, carbonyldiimidazole (CDI), 1-ethyl-3-(dimethylaminopropyl)carbodiimide (EDAC), chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)phosphoryl chloride (BOPCl) or sulfonyl chlorides, such as methane-sulfonyl chloride, toluenesulfonyl chloride or benzenesulfonyl chloride. According to one embodiment, a preferred activating agent is EDAC or DCC.

The reaction of VII with VIII or VIIIa is preferably carried out in the presence of a base. Suitable bases are the compounds listed for the cyclization of the dipeptide V to the piperazine IV. In one embodiment, the base used is triethylamine or N-ethyldiisopropylamine or a mixture thereof, particularly preferably N-ethyldiisopropylamine.

The deprotection of the compound VI or VIa to give the compound V or Va can be carried out by customary processes, such as, for example, according to Glenn L. Stahl et al., J. Org. Chem. 43(11), (1978), 2285-6 or A. K. Ghosh et al., Org. Lett. 3(4), (2001), 635-638. The deprotection is typically carried out by treatment with an acid. Suitable acids are both Brönstedt acids and Lewis acids, preferably organic carboxylic acids, for example formic acid, acetic acid or trifluoroacetic acid or mixtures thereof. In a preferred embodiment, the reaction is carried out in the presence of trifluoroacetic acid.

The reaction is usually carried out at temperatures in the range of from −30° C. to the boiling point of the reaction mixture, preferably from 0° C. to 50° C., particularly preferably from 20° C. to 35° C. The reaction can be carried out in a solvent, preferably in an inert organic solvent.

Suitable solvents are, in principle, the solvents mentioned above in connection with the basic cyclization of V to IV, in particular tetrahydrofuran or dichloromethane or mixtures thereof. In a preferred embodiment, the reaction is carried out in dichloromethane.

If another protective group is used instead of Boc, the deprotection method used will, of course, be suitable for the protective group in question.

If the groups R^{4a} and R^{5a} or R^{4c} and R^{5c} in the compounds IV and IVa are hydrogen, the compounds IV and IVa can also be prepared by intermolecular cyclization of a glycine ester derivative VIIa with a phenylalanine compound VIIIb or VIIIc according to the schemes below:

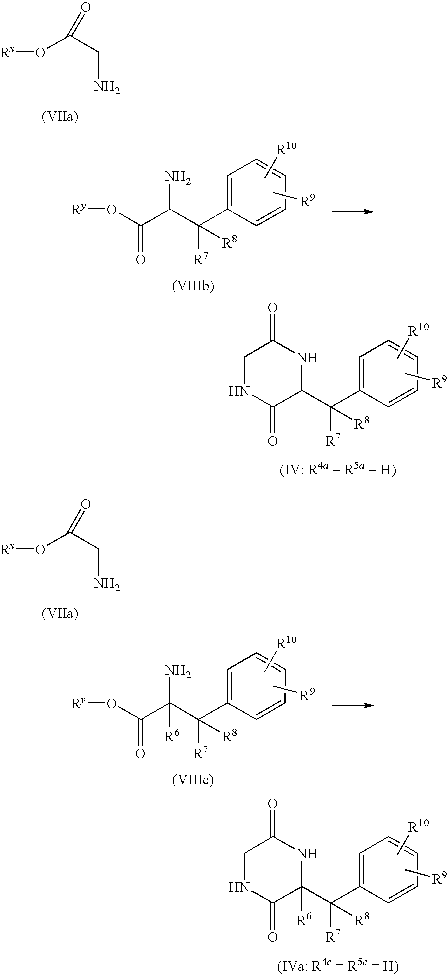

In the schemes, R^{x}, R⁶, R⁷, R⁸, R⁹ and R¹⁰ have the meanings given above. R^{y} is alkyl, for example methyl or ethyl. The intermolecular cyclization can be effected, for example, by a base, for example ammonia. The compounds VIIa and/or VIIIb or VIIIc can also be employed in the form of their acid addition salts, for example as hydrochlorides.

According to another embodiment (hereinbelow referred to as process B), the preparation of the compounds I comprises

i) providing a compound of the general formula IX

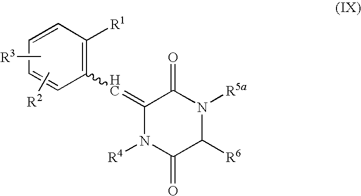

in which R¹, R², R³, R⁴ and R⁶ have the meanings mentioned above and R^{5a} has one of the meanings given for R⁵ different from hydrogen or is a protective group;
   ii) reacting the compound IX with the benzyl compound of the formula X

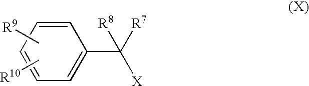

in which R⁷, R⁸, R⁹ and R¹⁰ have the meanings given above and X is a nucleophilically displaceable leaving group, in the presence of a base, which gives a compound IIb;

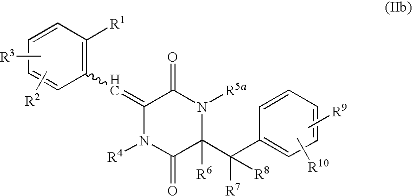

iii) hydrogenation of the resulting compound IIb
iv) if R^{5a} is a protective group, removing the protective group.

In formula IX, R^{5a} has preferably one of the meanings given for R⁵ different from hydrogen. In formula X, the variable X has preferably one of the following meanings: halogen, in particular chlorine, bromine or iodine, or O—SO₂—R^{m} where R^{m} has the meaning of C₁-C₄-alkyl or aryl which are optionally substituted by halogen, C₁-C₄-alkyl or halo-C₁-C₄-alkyl. Suitable protective groups for the nitrogen atoms of the piperazine rings in IX are in particular the radicals C(O)R⁵² mentioned above, for example the acetyl radical.

The reaction of the compound I× with the compound X in step ii) can be carried out analogously to the method described in process A, step iv) or, for example, according to the method described in J. Am. Chem. Soc. 105, 1983, 3214.

The hydrogenation in step ii) can likewise be carried out in the manner described above for the hydrogenation of compound II or IIa.

The compounds IX can be provided, for example, by reacting the compound X¹ with a benzaldehyde compound XII, as illustrated in the scheme below.

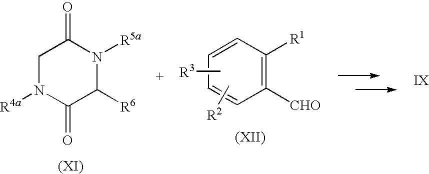

Here, R¹, R², R³, R^{5a} and R⁶ have the meanings mentioned above. R^{4a} has one of the meanings given above or is a protective group. Suitable protective groups for the nitrogen atoms of the piperazine ring in X¹ are in particular the radicals C(O)R⁵¹ mentioned above, for example the acetyl radical. R^{4a} and R^{5a} are in particular one of the radicals C(O)R⁵² mentioned above, for example acetyl radicals.

The reaction of X¹ with XII can be carried out under the conditions of an aldol condensation, as already described for the reaction of III with IV or IVa. Such aldol condensations can be carried out analogously to the processes described in J. Org. Chem. 2000, 65 (24), 8402-8405, Synlett 2006, 677, J. Heterocycl. Chem. 1988, 25, 591, which are hereby incorporated herein in their entirety.

The reaction is generally carried out in the presence of a base. The base used is preferably an alkali metal or alkaline earth metal carbonate, for example sodium carbonate, potassium carbonate or cesium carbonate, or mixtures thereof.

The reaction is preferably carried out in an inert, preferably aprotic organic solvent.

Examples of suitable solvents are in particular dichloromethane, dichloroethane, chlorobenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide, N-methylpyrrolidone and dimethylacetamide.

The compounds reacted are preferably those compounds XI in which R^{4a} and R^{5a} are a protective group and in particular an acyl radical R⁵²C(O)—(R⁵²═C₁-C₄-alkyl), for example an acetyl radical. Accordingly, the condensation reaction is generally followed by a removal of the protective groups. The removal of a protective group R^{4a}, R^{5a} can be carried out analogously to known processes of protective group chemistry, for example by the method described in Green, Wuts, Protective Groups in Organic Synthesis, 3rd ed. 1999, John Wiley and Sons, p. 553. This results in compounds of the formula IX in which R^{4a} and R^{5a} are hydrogen.

A subsequent alkylation for introducing the radicals R⁴ and/or R⁵ can be carried out by the method given above in process A for the alkylation of II, for example according to the methods described in Heterocycles, 45, 1997, 1151, and Chem. Commun. 1998, 659.

The compounds XI are known. Their preparation can be carried out analogously to the preparation of the compounds V described above, according to the scheme shown below:

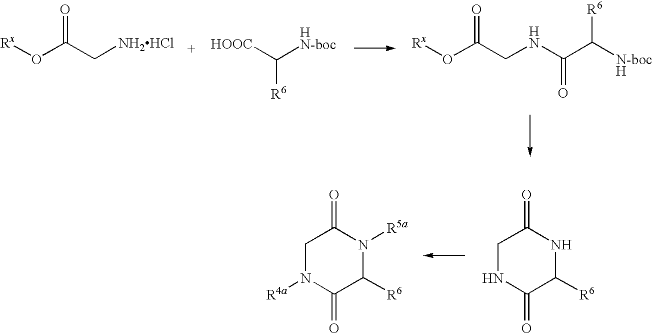

In this scheme, R^{4a}, R^{5a} and R⁶ have the meanings mentioned above. R^{x} is preferably C₁-C₄-alkyl or benzyl. Boc is a tert-butoxycarbonyl radical.

With respect to further details for the first reaction step, reference is made to the reaction of compound VII with the compound VIII or VIIIa or to the reaction of VIIa with VIIIb or \inc. The subsequent removal of the Boc protective group can be carried out analogously to the conversion of the compound VI into the compound V. The cyclization of the resulting deprotected compound can be carried out using the methods mentioned for the cyclization of the compound V. If R^{4a} and R^{5a} are a protective group, for example a radical C(O)R⁵¹, these protective groups can be introduced analogously to known processes of protective group chemistry, for example by reaction with anhydrides of the formula (R⁵¹C(O))₂O, for example by the method described in Green, Wuts, Protective Groups in Organic Synthesis, 3rd ed. 1999, John Wiley and Sons, p. 553.

According to a third process, the preparation of the compound I is carried out by cyclization of corresponding dipeptide precursors of the formula XIII, for example analogously to the method described by T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965. Hereinbelow, the cyclization of dipeptides of the formula XIII to the compounds according to the invention is also referred to as process C and is illustrated in the scheme below.

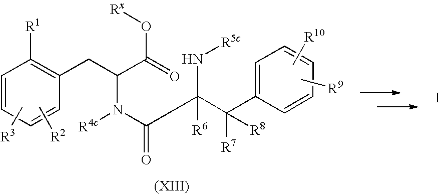

In formula XIII, the variables R¹, R², R³, R⁶, R⁷, R⁸, R⁹ and R¹⁰ have the meaning giving for formula I. R^{4c} is hydrogen or R⁴. R^{5c} is hydrogen, C₁-C₄-alkyl, C₃-C₄-alkenyl or C₃-C₄-alkynyl. The group OR^{x} is a suitable leaving group attached via oxygen. Here, R^{x} is, for example, hydrogen, C₁-C₆-alkyl, in particular methyl or ethyl, or phenyl-C₁-C₆-alkyl, for example benzyl. Dipeptides of the general formula XIII are novel and also form part of the subject matter of the present invention.

The cyclization can be carried out, for example, by reacting a dipeptide of the formula XIII either in the presence of acid or base (acidic or basic cyclization) or by heating the reaction mixture (thermal cyclization). With respect to the reaction conditions, reference is made to what was said for the cyclization of V to compound IV.

Preferably, R^{4c} and R^{5c} in formula XIII are hydrogen. In this case, subsequently to the cyclization an alkylation or acylation is carried out to introduce the radicals R⁴ and R⁵, respectively.

The preparation of dipeptide compounds XIII can be carried out analogously to the preparation of the compound V. The preparation is illustrated in the scheme below:

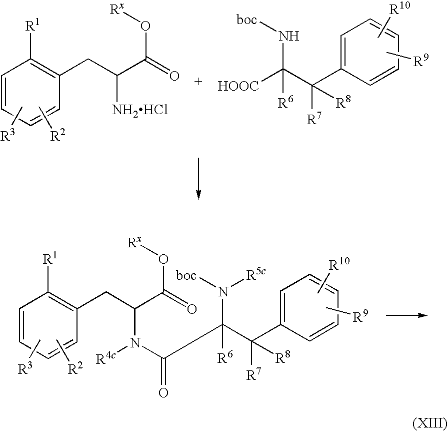

For further details about the first reaction step, reference is made to the reaction of compound VII with compound VIII. The subsequent removal of the boc protective group can be carried out analogously to the conversion of the compound VI into the compound V. The cyclization of the deprotected compound obtained in this manner can be carried out using the methods mentioned for the cyclization of the compound V.

The compounds of the formula I where R⁵≠H can also be prepared by reacting a piperazine compound of the formula I in which R⁵ is hydrogen with an alkylating agent or acylating agent which contains the radical R⁵ different from hydrogen. Such reactions can be carried out analogously to the methods discussed in connection with process A steps ii), iiia) and iv).

To this end, the piperazine compound of the formula I where R⁵=hydrogen is reacted with a suitable alkylating agent, hereinbelow compound X¹—R⁵, or acylating agent, hereinbelow compound X²—R⁵, which gives a piperazine compound of the formula I where R⁵≠hydrogen.

In the alkylating agents X¹—R⁵, X¹ can be halogen or O—SO₂—R^{m} where R^{m} has the meaning C₁-C₄-alkyl or aryl which are optionally substituted by halogen, C₁-C₄-alkyl or halo-C₁-C₄-alkyl. In acylating agents X²—R⁵, X² can be halogen, in particular Cl. Here, R⁵ is a radical (CO)R⁵¹.

The reaction is usually carried out at temperatures in the range of from −78° C. to the boiling point of the reaction mixture, preferably from −50° C. to 65° C., particularly preferably from −30° C. to 65° C. In general, the reaction is carried out in a solvent, preferably in an inert organic solvent.

Suitable solvents are the compounds mentioned for the cyclization of the dipeptide V to the piperazine IV, inter alia toluene, dichloromethane, tetrahydrofuran or dimethylformamide or mixtures thereof.

In a preferred embodiment, the compound I where R⁵═H is reacted with the alkylating or acylating agent in the presence of a base. Suitable bases are the compounds mentioned for the cyclization of the dipeptide V to the piperazine IV. The bases are generally employed in equimolar amounts. They can also be used in excess or even as solvent. In a preferred embodiment, the base is added in an equimolar amount or in an essentially equimolar amount. In a further preferred embodiment, the base used is sodium hydride.

Alternatively, the alkylation or acylation of the group NR⁵ in which R⁵ is H can also be carried out using the precursors. Thus, for example, compounds II, IV, V, VI, VIII in which R^{5a} or R^{5b} is H can be N-alkylated or N-acylated as described above. In the same manner, it is possible to alkylate the precursors II, IV, V, VI, VII in which the radical referred to as R⁴ or R^{4a} is hydrogen.

The compounds of the formula I can furthermore be modified at group R¹. Thus, for example, compounds of the formula I in which R¹ is CN, optionally substituted phenyl or an optionally substituted heterocyclic radical can be prepared from compounds I in which R¹ is halogen, such as chlorine, bromine or iodine, by conversion of the substituent R¹, for example analogously to the methods described by J. Tsuji, Top. Organomet. Chem. (14) (2005), 332 pp., J. Tsuji, Organic Synthesis with Palladium Compounds, (1980), 207 pp., Tetrahedron Lett. 42, 2001, p. 7473 or Org. Lett. 5, 2003, 1785.

To this end, a piperazine compound of the formula I which, as substituent R¹, has a halogen atom, such as chlorine, bromine or iodine, can be converted by reaction with a coupling partner which contains a group R¹ (compound R¹—X³) into another piperazine derivative of the formula I. In an analogous manner, it is also possible to react the compounds Ia, II and IIa.

The reaction is usually carried out in the presence of a catalyst, preferably in the presence of a transition metal catalyst. In general, the reaction is carried out in the presence of a base.

Suitable coupling reagents X³—R¹ are in particular those compounds in which X³, if R¹ is phenyl or a heterocyclic radical (heterocyclyl), denotes one of the following groups:
Zn—R^{l} where R^{l} is halogen, phenyl or heterocyclyl;
B(OR^{m})₂, where R^{m} is H or C₁-C₆-alkyl, where two alkyl substituents together may form a C₂-C₄-alkylene chain; or
SnRⁿ₃ where Rⁿ is C₁-C₆-alkyl.

This reaction is usually carried out at temperatures in the range from −78° C. to the boiling point of the reaction mixture, preferably from −30° C. to 65° C., particularly preferably at temperatures from 30° C. to 65° C. In general, the reaction is carried out in an inert organic solvent in the presence of a base.

Suitable solvents are the compounds mentioned in connection with the cyclization of the dipeptide IV to the piperazine V. In one embodiment of the process according to the invention, use is made of tetrahydrofuran with a catalytic amount of water; in another embodiment, only tetrahydrofuran is used.

Suitable bases are the compounds mentioned for the cyclization of the dipeptide IV to the piperazine V.

The bases are generally employed in equimolar amounts. They can also be employed in excess or even as solvent.

In a preferred embodiment of the process according to the invention, the base is added in an equimolar amount. In a further preferred embodiment, the base used is triethylamine or cesium carbonate, particularly preferably cesium carbonate.

Suitable catalysts for the process according to the invention are, in principle, compounds of the transition metals Ni, Fe, Pd, Pt, Zr or Cu. It is possible to use organic or inorganic compounds. Pd(PPh₃)₂Cl₂, Pd(OAc)₂, PdCl₂ or Na₂PdCl₄ may be mentioned by way of example. Here, Ph is phenyl; Ac is Acetyl.

The different catalysts can be employed either individually or else as mixtures. In a preferred embodiment of the invention, Pd(PPh₃)₂Cl₂ is used.

To prepare the compound I in which R¹ is CN, the compound I in which R¹ is chlorine, bromine or iodine can also be reacted with copper cyanide, analogously to known processes (see, for example, Organikum, 21. edition, 2001, Wiley, p. 404, Tetrahedron Lett. 42, 2001, p. 7473 or Org. Lett. 5, 2003, 1785 and the literature cited therein).

These conversions are usually carried out at temperatures in the range of from 100° C. to the boiling point of the reaction mixture, preferably at from 100° C. to 250° C. In general, the reaction is carried out in an inert organic solvent. Suitable solvents are in particular aprotic polar solvents, for example dimethylformamide, N-methylpyrrolidone, N,N′-dimethylimidazolidin-2-one and dimethylacetamide.

Alternatively, the conversion of group R¹ can also be carried out on the precursors of the compound I. Thus, for example, compounds II in which R¹ is a halogen atom such as chlorine, bromine or iodine can be subjected to the reaction described above.

Alternatively, the alkylation or acylation of the group NR^{4a}, NR^{5a} in which R^{4a} or R^{5a} is H can also be carried out using the precursors, Thus, for example, compounds II, IV, V, VI, VIII in which R^{5a} or R^{5b} is H can be N-alkylated or N-acylated as described above. In the same manner, it is possible to alkylate the precursors II, IV, V, VI, VII in which the radical referred to as R⁴ or R^{4a} is hydrogen.

The compounds I and their agriculturally useful salts are suitable, both in the form of isomer mixtures and in the form of the pure isomers, as herbicides. They are suitable as such or as an appropriately formulated composition. The herbicidal compositions comprising the compound I or Ia control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds I or Ia, or compositions comprising them, can additionally be employed in a further number of crop plants for eliminating undesirable plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

Preferred crops are the following: Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N. rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (s. vulgare), Triticale, Triticum aestivum, Triticum durum, Vicia faba, Vitis vinifera and Zea mays.

In addition, the compounds of the formula I may also be used in crops which tolerate the action of herbicides owing to breeding, including genetic engineering methods.

In addition, the compounds of the formula I can also be used in crops which tolerate insects or fungal attack as the result of breeding, including genetic engineering methods.

Furthermore, it has been found that the compounds of the formula I are also suitable for the defoliation and/or desiccation of plant parts, for which crop plants such as cotton, potato, oilseed rape, sunflower, soybean or field beans, in particular cotton, are suitable. In this regard, there have been found compositions for the desiccation and/or defoliation of plants, processes for preparing these compositions and methods for desiccating and/or defoliating plants using the compounds of the formula I.

As desiccants, the compounds of the formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

The compounds I, or the herbicidal compositions comprising the compounds I, can be used, for example, in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading, watering or treatment of the seed or mixing with the seed. The use forms depend on the intended purpose; in any case, they should ensure the finest possible distribution of the active ingredients according to the invention.

The herbicidal compositions comprise a herbicidally effective amount of at least one compound of the formula I or an agriculturally useful salt of I, and auxiliaries which are customary for the formulation of crop protection agents.

Examples of auxiliaries customary for the formulation of crop protection agents are inert auxiliaries, solid carriers, surfactants (such as dispersants, protective colloids, emulsifiers, wetting agents and tackifiers), organic and inorganic thickeners, bactericides, antifreeze agents, antifoams, optionally colorants and, for seed formulations, adhesives.

Examples of thickeners (i.e. compounds which impart to the formulation modified flow properties, i.e. high viscosity in the state of rest and low viscosity in motion) are polysaccharides, such as xanthan gum (Kelzan® from Kelco), Rhodopol® 23 (Rhone Poulenc) or Veegum® (from R. T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia), long-chain alcohols, fatty acids, salts of fatty acids, organofluorine compounds and mixtures thereof.

Bactericides can be added for stabilizing the aqueous herbicidal formulations. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert auxiliaries are, for example, the following:

mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone or strongly polar solvents, for example amines such as N-methylpyrrolidone, and water.

Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF AG), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denaturated proteins, polysaccharides (e.g. methylcellulose), hydrophobically modified starches, polyvinyl alcohol (Mowiol types Clariant), polycarboxylates (BASF AG, Sokalan types), polyalkoxylates, polyvinylamine (BASF AG, Lupamine types), polyethyleneimine (BASF AG, Lupasol types), polyvinylpyrrolidone and copolymers thereof.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water. To prepare emulsions, pastes or oil dispersions, the compounds of the formula I or Ia, either as such or dissolved in an oil or solvent, can be homogenized in water by means of a wetting agent, tackifier, dispersant or emulsifier. Alternatively, it is also possible to prepare concentrates comprising active compound, wetting agent, tackifier, dispersant or emulsifier and, if desired, solvent or oil, which are suitable for dilution with water.

The concentrations of the compounds of the formula I in the ready-to-use preparations can be varied within wide ranges. In general, the formulations comprise approximately from 0.001 to 98% by weight, preferably 0.01 to 95% by weight of at least one active ingredient. The active ingredients are employed in a purity of from 90% to 100%, preferably 95% to 100% (according to NMR spectrum).

The compounds I of the invention can for example be formulated as follows:

1. Products for Dilution with Water

### A Water-Soluble Concentrates

10 parts by weight of active compound are dissolved in 90 parts by weight of water or a water-soluble solvent. As an alternative, wetters or other adjuvants are added. The active compound dissolves upon dilution with water. This gives a formulation with an active compound content of 10% by weight.

### B Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight.

### C Emulsifiable Concentrates

15 parts by weight of active compound are dissolved in 75 parts by weight of an organic solvent (eg. alkylaromatics) with addition of calcium dodecylbenzenesulfonate and castor oil ethoxylate (in each case 5 parts by weight). Dilution with water gives an emulsion. The formulation has an active compound content of 15% by weight.

### D Emulsions

25 parts by weight of active compound are dissolved in 35 parts by weight of an organic solvent (eg. alkylaromatics) with addition of calcium dodecylbenzenesulfonate and castor oil ethoxylate (in each case 5 parts by weight). This mixture is introduced into 30 parts by weight of water by means of an emulsifier (Ultraturrax) and made into a homogeneous emulsion. Dilution with water gives an emulsion. The formulation has an active compound content of 25% by weight.

### E Suspensions

In an agitated ball mill, 20 parts by weight of active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules

50 parts by weight of active compound are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders

75 parts by weight of active compound are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations

In a ball mill, 20 parts by weight of active compound, 10 parts by weight of dispersant, 1 part by weight of gelling agent and 70 parts by weight of water or of an organic solvent are mixed to give a fine suspension. Dilution with water gives a stable suspension with active compound content of 20% by weight.

### 2. Products to be Applied Undiluted

### I Dusts

5 parts by weight of active compound are ground finely and mixed intimately with 95 parts by weight of finely divided kaolin. This gives a dusting powder with an active compound content of 5% by weight.

### J Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

### K ULV Solutions (UL)

10 parts by weight of active compound are dissolved in 90 parts by weight of an organic solvent, for example xylene. This gives a product to be applied undiluted with an active compound content of 10% by weight.

The compounds of the formula I or the herbicidal compositions comprising them can be applied pre- or post-emergence, or together with the seed of a crop plant. It is also possible to apply the herbicidal composition or active compounds by applying seed, pretreated with the herbicidal compositions or active compounds, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the compounds of the formula I or the herbicidal compositions can be applied by treating seed.

The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the compounds of the formula I according to the invention or the compositions prepared therefrom. Here, the herbicidal compositions can be applied diluted or undiluted.

The term seed comprises seed of all types, such as, for example, corns, seeds, fruits, tubers, seedlings and similar forms. Here, preferably, the term seed describes corns and seeds.

The seed used can be seed of the useful plants mentioned above, but also the seed of transgenic plants or plants obtained by customary breeding methods.

The rates of application of the active compound are from 0.001 to 3.0, preferably 0.01 to 1.0, kg/ha of active substance (a.s.), depending on the control target, the season, the target plants and the growth stage. To treat the seed, the compounds I are generally employed in amounts of from 0.001 to 10 kg per 100 kg of seed.

To widen the spectrum of action and to achieve synergistic effects, the compounds of the formula I may be mixed with a large number of representatives of other herbicidal or growth-regulating active ingredient groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, 2-hetaroyl-1,3-cyclohexane-diones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils, phenyl pyrazolines and isoxazolines and derivatives thereof.

It may furthermore be beneficial to apply the compounds of the formula I alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

Moreover, it may be useful to apply the compounds of the formula I in combination with safeners. Safeners are chemical compounds which prevent or reduce damage on useful plants without having a major impact on the herbicidal action of the compounds of the formula I towards unwanted plants. They can be applied either before sowings (e.g. on seed treatments, shoots or seedlings) or in the pre-emergence application or post-emergence application of the useful plant. The safeners and the compounds of the formula I can be applied simultaneously or in succession. Suitable safener are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenyl-carbamates and their agriculturally acceptable salts and their agriculturally acceptable derivatives such amides, esters, and thioesters, provided they have an acid group.

Hereinbelow, the preparation of piperazine compounds of the formula I is illustrated by examples; however, the subject matter of the present invention is not limited to the examples given.

### EXAMPLES

The products shown below were characterized by determination of the melting point, by NMR spectroscopy or by the masses determined by HPLC-MS spectrometry ([m/z]) or by the retention time (RT; [min.]).

[HPLC-MS=high performance liquid chromatography coupled with mass spectrometry; HPLC column: RP-18 column (Chromolith Speed ROD from Merck KgaA, Germany), 50×4,6 mm; mobile phase: acetonitrile+0.1% trifluoroacetic acid (TFA)/water+0.1% TFA, gradient from 5:95 to 100:0 over 5 minutes at 40° C., flow rate 1.8 ml/min;

MS: quadrupole electrospray ionisation, 80 V (positive mode)].

### Example 1

### 2-[5-Benzyl-1,4,5-trimethyl-3,6-dioxopiperazine-2-ylmethyl]benzonitrile

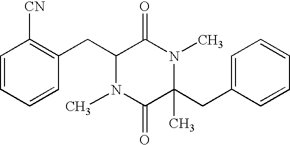

### 1.1 Preparation of methyl (2-tert-butoxycarbonylamino-3-phenylpropionylamino)-acetate

At 0° C., ethyldiisopropylamine (259 g, 2.0 mol), N-tert-butoxycarbonyl-L-phenyl-alanine (212 g, 0.8 mol) and 1-ethyl-3-(3′-dimethylaminopropyl)carbodiimide (EDAC, 230 g, 1.2 mol) were added to a solution of glycine methyl ester hydrochloride (100 g, 0.8 mol) in tetrahydrofuran (THF, 1000 ml). The reaction mixture was then stirred at room temperature for 24 h. The reaction mixture obtained was freed under reduced pressure from volatile components, and the residue obtained in this manner was taken up in water (1000 ml). The aqueous phase was extracted repeatedly with CH₂Cl₂. The organic phases obtained in this manner were combined, washed with water, dried over Na₂SO₄, filtered and freed from the solvent under reduced pressure. Methyl (2-tert-butoxycarbonylamino-3-phenylpropionylamino)acetate was obtained as a yellow oil in an amount of 300 g. The crude product obtained was reacted further without further purification.

### 1.2 Preparation of 3-benzylpiperazine-2,5-dione

At room temperature, trifluoroacetic acid (342 g, 3 mol) was added dropwise to a solution of methyl (2-tert-butoxycarbonylamino-3-phenylpropionylamino)acetate (300 g, about 0.8 mol) in CH₂Cl₂. The reaction mixture obtained was stirred at room temperature for 24 h and then concentrated under reduced pressure. The residue obtained was taken up in THF (500 ml), and an aqueous solution of ammonia (25% strength, 500 ml) was added slowly. The reaction mixture was stirred at room temperature for a further 72 h. The precipitated solid was isolated by filtration and washed with water. 3-Benzylpiperazine-2,5-dione was obtained in an amount of 88 g (yield 54%).

### 1.3 Preparation of 1,4-diacetyl-3-benzyl-piperazine-2,5-dione

A solution of 3-benzylpiperazine-2,5-dione (20.4 g, 0.1 mol) in acetic anhydride (200 ml) was stirred under reflux conditions for 4 h. The reaction mixture obtained was concentrated under reduced pressure. The residue was taken up in CH₂Cl₂, washed successively with an aqueous NaHCO₃ solution and water, dried over Na₂SO₄, filtered and freed from the solvent under reduced pressure. 1,4-Diacetyl-3-benzylpiperazine-2,5-dione was obtained as a yellow oil in an amount of 28.5 g (quantitative) and reacted further as crude product.

HPLC-MS [m/z]: 289.1 [M+1]⁺.

### 1.4 Preparation of 1-acetyl-6-benzyl-3-(2-bromobenzylidene)piperazine-2,5-dione

Bromobenzaldehyde (5.55 g, 0.03 mol) and Cs₂CO₃ (9.8 g, 0.03 mol) were added to a solution of 1,4-diacetyl-3-benzylpiperazine-2,5-dione (17.4 g, 0.06 mol) in dimethylformamide (DMF, 100 ml). The reaction mixture was stirred at room temperature for 36 h, water (500 ml) and citric acid (10 g) were then added and the mixture was extracted repeatedly with CH₂Cl₂. The organic phases obtained in this manner were combined, washed with water, dried over Na₂SO₄, filtered and freed from the solvent under reduced pressure. After purification by column chromatography (mobile phase: CH₂Cl₂), 1-acetyl-6-benzyl-3-(2-bromobenzylidene)piperazine-2,5-dione was obtained as a yellow oil in an amount of 12 g (yield 48%).

HPLC-MS [m/z]: 413.9 [M+1]⁺.

### 1.5 Preparation of 3-benzyl-6-(2-bromobenzylidene)-piperazine-2,5-dione

Dilute aqueous HCl solution (5% strength, 250 ml) was added to a solution of 1-acetyl-6-benzyl-3-(2-bromobenzylidene)piperazine-2,5-dione (12 g, 0.03 mol) in THF (50 ml). The reaction mixture was stirred under reflux conditions for 8 h. After cooling of the reaction solution, the precipitated solid was isolated by filtration. The solid obtained in this manner was washed with water and THF. 3-Benzyl-6-(2-bromobenzylidene)piperazine-2,5-dione was obtained as a colorless solid in an amount of 8.3 g (yield 75%).

HPLC-MS [m/z]: 371.2 [M]⁺.

### 1.6 3-Benzyl-6-(2-bromobenzylidene)-1,3,4-trimethylpiperazine-2,5-dione

At 0° C., NaH (0.85 g, 60% pure, 21 mmol) was added to a solution of 3-benzyl-6-(2-bromobenzylidene)piperazine-2,5-dione (2.00 g, 5.4 mmol) in DMF (50 ml). The reaction mixture was stirred at 0° C. for 2 h, and MeI (5.0 g, 35 mmol) was then added. The reaction mixture was stirred at room temperature for a further 18 h, and water was then added. The mixture was extracted repeatedly with methyl tert-butyl ether. The organic phases obtained in this manner were combined, washed with water, dried over Na₂SO₄, filtered and freed from the solvent under reduced pressure. After purification by column chromatography, 3-benzyl-6-(2-bromobenzylidene)-1,3,4-trimethylpiperazine-2,5-dione was obtained in an amount of 1.6 g (yield 72%).

HPLC-MS [m/z]: 413.0 [M]⁺.

### 1.7 Preparation of 2-(5-benzyl-1,4,5-trimethyl-3,6-dioxopiperazin-2-ylidenemethyl)-benzonitrile

CuCN (0.7 g, 7.8 mmol) was added to a solution of 3-benzyl-6-(2-bromo-benzylidene)-1,3,4-trimethylpiperazine-2,5-dione (1.5 g, 3.6 mmol) in N-methylpyrrolidin (NMP, 25 ml). The reaction mixture was stirred at 155 C. for 16 h and, after cooling to room temperature, introduced into ethyl acetate. The reaction mixture was diluted with methyl tert-butyl ether. The organic phase obtained in this manner was washed with water, dried over Na₂SO₄, filtered and freed from the solvent under reduced pressure. Purification by column chromatography gave 2-(5-benzyl-1,4,5-trimethyl-3,6-dioxopiperazin-2-ylidenemethyl)benzonitrile in an amount of 0.79 g (yield 61%).

HPLC-MS [m/z]: 360.5 [M+1]⁺.

### 1.8 Preparation of 2-[5-benzyl-1,4,5-trimethyl-3,6-dioxopiperazin-2-ylmethyl)benzonitrile

Under nitrogen, Pd on activated carbon (0.1 g) as a suspension in MeOH (2 ml) was added to a solution of 2-(5-benzyl-1,4,5-trimethyl-3,6-dioxopiperazin-2-ylidenemethyl)benzonitrile (0.5 g, 1.4 mmol) in MeOH (methanol, 40 ml). The resulting suspension was hydrogenated under an H₂ atmosphere for 7 h. The reaction mixture obtained was filtered through Celite. The filtrate was freed from the solvent under reduced pressure. The crude product obtained in this manner was purified by column chromatography. This gave 2 isomers which were examined by HPLC-MS.

Main isomer 1: HPLC-MS: [m/z]=362.1 [M+H]*; RT=2.834 min;

Minor isomer 2: HPLC-MS: [m/z]=362.1 [M+H]*; RT=2.657 min.

### Example 2

### Alternative preparation of 2-[5-benzyl-1,4,5-trimethyl-3,6-dioxopiperazin-2-ylmethyl)benzonitrile

### 2.1 Preparation of ethyl N-(diphenylmethylene)glycinate

Ethyl glycinate hydrochloride (37 g, 0.27 mol) was dissolved in a solution of K₂CO₃ (74.4 g, 0.54 mol) in water (186 ml). The solution was stirred for 15 min and then extracted with dichloromethane (10*150 ml). The organic phases obtained in this manner were combined, dried over MgSO₄ and freed from solvent under reduced pressure (500 mbar) (yield ˜50%). The residue (9.5 g, 0.092 mol) was, together with benzophenone (14.03 g, 0.077 mol) dissolved in xylene (76 ml). After addition of a few drops of BF₃*Et₂₀, the reaction mixture was stirred under reflux conditions on a water separator for 5 h. After cooling of the reaction mixture to room temperature, the solvent was removed under reduced pressure.

From the residue obtained, ethyl N-(diphenylmethylene)glycinate was isolated by distillation (80° C. at 5.5*10⁻² mbar) in a yield of 48%.

### 2.2 Preparation of ethyl N-(diphenylmethylene)-α-(2-cyanophenyl)alaninate

Aqueous sodium hydroxide solution NaOH (10% strength, 40 ml) was added to a solution of ethyl N-(diphenylmethylene)glycinate (5 g, 18.7 mmol; from Example 2.1), 2-cyanobenzyl bromide (4.1 g, 20.7 mmol) and tetrabutylammonium sulfate (320 m, 0.9 mmol) in dichloromethane (40 ml), and the mixture was stirred at room temperature overnight. The phases were separated, and the aqueous phase was then extracted with dichloromethane (2 times 50 ml). The organic phases obtained were combined, washed with water until the wash phase remained neutral, dried over MgSO₄, filtered and freed from the solvent under reduced pressure. From the residue obtained, ethyl N-(diphenylmethylene)-α-(2-cyanophenyl)alaninate was isolated by flash chromatography (SiO₂; cyclohexane/ethyl acetate) in a yield of 83%.

### 2.3 Preparation of ethyl α-(2-cyanophenyl)alaninate hydrochloride

Aqueous HCl (1M, 95 ml) was added to a solution of ethyl N-(diphenyl-methylene)-α-(2-cyanophenyl)alaninate (11.4 g, 29.8 mmol; from Example 2.2) in acetone (95 ml). The mixture was stirred at room temperature for 3 h and then freed from the solvent under reduced pressure. Diethyl ether (2 times 50 ml) was added to the residue obtained. The supernatant liquid was decanted off. The solid that remains is ethyl α-(2-cyanophenyl)alaninate hydrochloride which can be used without further purification for the next step (yield 87%).

### 2.4 Preparation of N-(tert-butoxycarbonyl)-α-methylphenylalanine

Aqueous sodium hydroxide solution (1M, 170 ml) was added to a suspension of α-methylphenylalanine (20 g, 0.11 mol) in dioxane/water (2:1, 300 ml). At a temperature of 0° C., a solution of di-tert-butyl dicarbonate (29.2 g, 0.134 mol) in dioxane (50 ml) was slowly added dropwise to this reaction mixture. After the addition had ended, the reaction mixture was stirred at room temperature overnight. The reaction was monitored by LC-MS analysis. In each case half an equivalent of di-tert-butyl dicarbonate was added until no more starting material could be detected. In each case, the pH was adjusted to 9 using aqueous sodium hydroxide solution NaOH (1M). Using 10% strength aqueous hydrochloric acid, the reaction mixture was then adjusted to a pH of 2 and extracted with ethyl acetate. The organic phases obtained were combined, washed with water, dried over MgSO₄, filtered and freed from the solvent under reduced pressure. The N-(tert-butoxycarbonyl)-α-methylphenylalanine obtained as a residue in a yield of 88% can be used without further purification for the next step.

### 2.5 Preparation of (N-Boc-α-CH₃-Phe)-(o-CN-Phe)-OC₂H₅

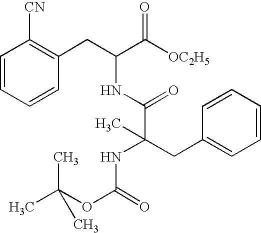

At 0° C. and under an N₂ atmosphere, a solution of N-(tert-butoxycarbonyl)-α-methylphenylalanine (6.3 g, 22.6 mmol)) in tetrahydrofuran (THF, 13 ml) was added to a suspension of N,N′-carbonyldiimidazole (CDI, 3.7 g, 27.1 mmol) in THF (34 ml). The reaction mixture was stirred at room temperature for 8 h. Ethyl α-(2-cyanophenyl)alaninate hydrochloride (8.6 g, 33.8 mmol) was then added a little at a time, followed by diisopropylethylamine (DIPEA, 8.7 g, 67.6 mmol). The reaction mixture was stirred at 45° C. overnight and then under reflux conditions for 2 h. The reaction mixture was put onto aqueous 5% strength citric acid and then extracted with ethyl acetate. The organic phases obtained were combined, washed with saturated aqueous NaHCO₃ solution, dried over MgSO₄, filtered and freed from the solvent under reduced pressure. (N-Boc-α-CH₃-Phe)-(o-CN-Phe)-OC₂H₅ was obtained from the residue by flash chromatography (SiO₂, cyclohexane/ethyl acetate) in a yield of about 40%.

### 2.6 Preparation of (α-CH₃-Phe)-(o-CN-Phe)-OH

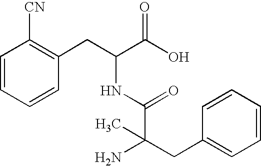

Trifluoroacetic acid (TFA, 8.20 g, 71.9 mmol) was added to a solution of (N-Boc-α-CH₃-Phe)-(o-CN-Phe)-OC₂H₅ (4.1 g, 8.5 mmol) in dichloromethane (14 ml). The reaction mixture was stirred at room temperature for 2 h and then freed from volatile components under reduced pressure. The residue was taken up in chloroform. The reaction mixture was washed with saturated aqueous Na₂CO₃ solution. The organic phase was dried over MgSO₄, filtered and freed from the solvent under reduced pressure. The residue obtained (˜1 g) was, at a temperature of 0° C., taken up in a mixture of tetrahydrofuran/aqueous sodium hydroxide solution (2M) (1:1, 10 ml). The mixture was stirred at this temperature for 2 h. The pH was then adjusted to 7 using hydrochloric acid (10% strength). The mixture was washed with ethyl acetate. The aqueous phase obtained was dried under reduced pressure. The residue consisted of (α-CH₃-Phe)-(o-CN-Phe)-OH and salts originating from the neutralization. Yield: 1.2 g (<40%).

### 2.7 Preparation of 2-(5-benzyl-3,6-dioxo-5-methylpiperazin-2-ylmethyl)benzonitrile

Under an N2 atmosphere, a suspension of (α-CH₃-Phe)-(o-CN-Phe)-OH (0.92 g, 2.6 mmol) and di(N-succinimidyl) carbonate (0.8 g, 3.1 mmol) in dry acetonitrile (35 ml) was stirred at room temperature for 12 h. Diisopropylethylamine (DIPEA, 0.47 ml, 2.6 mmol) was then added to the reaction mixture. The reaction mixture was stirred at room temperature for a further 12 h. The solvent was removed under reduced pressure. The residue was taken up in water (2×5 ml) and stirred. The precipitated solid was isolated by filtration. From the solid, 2-(5-benzyl-3,6-dioxo-5-methylipiperazin-2-ylmethyl)benzonitrile was isolated by preparative HPLC chromatography (RP; mobile phase: water/acetonitrile) in an amount of 315 mg (yield 36%).

### 2.8 Preparation of 2-(5-benzyl-1,4,5-trimethyl-3,6-dioxopiperazin-2-ylmethyl)benzonitrile

At 0° C. and under an N2 atmosphere, NaH (144 mg, 3.6 mmol) was added to a solution of 2-(5-benzyl-3,6-dioxo-5-methylpiperazin-2-ylmethyl)benzonitrile (0.3 g, 0.9 mmol) in dry dimethylformamide (DMF), and the mixture was stirred at this temperature for 1 h. MeI (0.77 g, 5.4 mmol) was then added. The reaction mixture was stirred at room temperature for one hour, and the solvent was then removed under reduced pressure. The residue obtained was separated by preparative HPLC chromatography (RP; mobile phase: water/acetonitrile). 2-(5-Benzyl-1,4,5-trimethyl-3,6-dioxopiperazin-2-ylmethyl)benzonitrile was obtained in an amount of 77 mg as a mixture of two diastereomers. The diastereomers were isolated by preparative thin-layer chromatography (SiO₂, cyclohexane/ethyl acetate 1:3). The first diastereomer was obtained in an amount of 6 mg (R_{f}=0.25). The second diastereomer was obtained in an amount of 24 mg (R_{f}=0.12). This corresponds to a yield of 10%.

### Example 3

### 3-Benzyl-6-(2,3-dichlorobenzyl)-1,3,4-trimethylpiperazine-2,5-dione

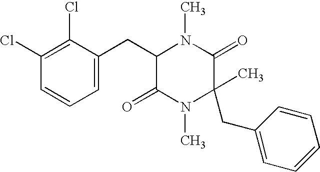

### 3.1 Preparation of 3-benzyl-1,3,4-trimethylpiperazine-2,5-dione

At 0° C., NaH (3.96 g, 60%, 0.1 mol) was added, in portions, to a solution of 3-benzyl-3-methyl-piperazine-2,5-dione (9.8 g, 0.045 mol) in dimethylformamide (450 mL). The reaction mixture was stirred at 0° C. for 30 minutes and methyl iodide (14.05 g, 0.1 mol) was added. The reaction mixture was stirred at room temperature for a further hour, and then a saturated solution of NH₄OH was added. The mixture was extracted repeatedly with CH₂Cl₂. The organic phases obtained in this manner were combined, washed with water, dried over Na₂SO₄, filtered and freed from the solvent under reduced pressure. The solid obtained in this manner was washed with cold CH₂Cl₂. 3-Benzyl-1,3,4-trimethylpiperazine-2,5-dione was obtained as a bright solid in an amount of 9.6 g (yield 87%).

HPLC-MS [m/z]: 247.1 [M+H]⁺

### 3.2 Preparation of 3-benzyl-6-(2,3-dichlorobenzyl)-1,3,4-trimethylpiperazine-2,5-dione

Under argon, 35 mg of NaH (60%) were added to a solution of 170 mg (0.7 mmol) of 3-benzyl-1,3,4-trimethylpiperazine-2,5-dione in 2 mL of dimethylformamide (DMF). The reaction mixture was stirred at room temperature for 10 minutes. 167 mg (0.7 mmol) of 1-bromomethyl-2,3-dichlorobenzene were added and stirring was continued at room temperature for 48 hours. Water was added and the reaction mixture was extracted with dichloromethane. After purification of the crude product by preparative HPLC, the title compound was obtained in a yield of 20%.

The preparation of the compounds of the formula I compiled in Table 1 (Examples 4 to 39) was carried out analogously to Examples 1, 2 and 3 shown above.

TABLE 1
Compounds of the general formula I, wherein R⁴ is
CH₃ and R⁷, R⁸, R⁹ and R¹⁰ and each hydrogen

| | |
|---|---|
| | (I) |
| 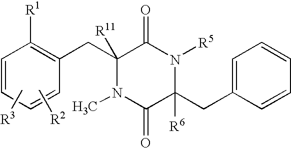 | |

| | | | | | | | mp. [° C.] and/or |
|---|---|---|---|---|---|---|---|
| No. | R¹ | R² | R³ | R⁵ | R⁶ | R¹¹ | RT, [m/z] |
| 1^{*)} | CN | H | H | CH₃ | CH₃ | H | 2.834 min |
| | | | | | | | m/z = 362.1 [M + H] |
| 1^{**)} | CN | H | H | CH₃ | CH₃ | H | 2.657 min |
| | | | | | | | m/z = 362.1 [M + H] |
| 4 | CN | H | H | CH₃ | CH₃ | CH₃ | 130-132° C. |
| | | | | | | | 2.968 min |
| | | | | | | | m/z = 376.1 [M + H] |
| 5 | NO₂ | H | H | CH₃ | CH₃ | H | 3.245 min |
| | | | | | | | m/z = 382.8 [M + H] |
| 6 | Cl | H | H | CH₃ | CH₃ | H | 3.635 min |
| | | | | | | | m/z = 372.3 [M + H] |
| 7 | Cl | 6-Cl | H | CH₃ | CH₃ | H | 3.785 min |
| | | | | | | | m/z = 406.1 [M + H] |
| 8 | NO₂ | 6-Cl | H | CH₃ | CH₃ | H | 3.606 min |
| | | | | | | | m/z = 416.12 [M + H] |
| 9 | Cl | 5-CF₃ | H | CH₃ | CH₃ | H | 3.580 min |
| | | | | | | | m/z = 439.3 [M + H] |
| 10 | F | 5-Cl | H | CH₃ | CH₃ | H | 3.695 min |
| | | | | | | | m/z = 390.9 [M + H] |
| 11 | Br | 5-F | H | CH₃ | CH₃ | H | 3.733 min |
| | | | | | | | m/z = 436.12 [M + 2] |
| 12 | F | 3-Cl | H | CH₃ | CH₃ | H | 3.700 min |
| | | | | | | | m/z = 390.2 [M + H] |
| 13 | F | 5-F | 6-Cl | CH₃ | CH₃ | H | 3.642 min |
| | | | | | | | m/z = 408.1 [M + H] |
| 14 | F | 5-CH₃ | 6-F | CH₃ | CH₃ | H | 3.686 min |
| | | | | | | | m/z = 388.3 [M + 2] |
| 15 | F | 5-CF₃ | H | CH₃ | CH₃ | H | 3.758 min |
| | | | | | | | m/z = 424.2 [M + 2] |
| 16 | F | 3-F | H | CH₃ | CH₃ | H | 3.501 min |
| | | | | | | | m/z = 474.2 [M + 2] |
| 17 | F | 5-CF₃ | 3-Cl | CH₃ | CH₃ | H | 4.041 min |
| | | | | | | | m/z = 458.3 [M + H] |
| 18 | F | 3-CF₃ | H | CH₃ | CH₃ | H | 3.754 min |
| | | | | | | | m/z = 424.2 [M + 2] |
| 19 | F | 5-CH₃ | 6-Cl | CH₃ | CH₃ | H | 3.901 min |
| | | | | | | | m/z = 404.2 [M + 2] |
| 20 | Br | H | H | CH₃ | CH₃ | H | 3.777 min |
| | | | | | | | m/z = 415.8 [M + H] |
| 21 | F | H | H | CH₃ | CH₃ | H | 3.641 min |
| | | | | | | | m/z = 355.9 [M + H] |
| 22 | F | 4-F | H | CH₃ | CH₃ | H | 3.189 min |
| | | | | | | | m/z = 372.9 [M + H] |
| 23 | NO₂ | 3-Cl | H | CH₃ | CH₃ | H | 3.663 min |
| | | | | | | | m/z = 416.9 [M + H] |
| 24 | Cl | 3-CH(CH₃)₂ | H | CH₃ | CH₃ | H | 4.346 min |
| | | | | | | | m/z = 413.9 [M + H] |
| 25 | Cl | 3-CF₃ | H | CH₃ | CH₃ | H | 4.024 min |
| | | | | | | | m/z = 439.8 [M + H] |
| 26 | Cl | 4-F | H | CH₃ | CH₃ | H | 3.629 min |
| | | | | | | | m/z = 390.2 [M + 2] |
| 27 | F | 5-F | H | CH₃ | CH₃ | H | 3.014 min |
| | | | | | | | m/z = 373.1 [M + H] |
| 28 | F | 6-F | H | CH₃ | CH₃ | H | 3.384 min |
| | | | | | | | m/z = 373.8 [M + H] |
| 29 | Cl | 4-OCH₃ | 3-Cl | CH₃ | CH₃ | H | 3.161 min |
| | | | | | | | m/z = 437.7 [M + 2] |
| 30 | F | 4-F | 6-F | CH₃ | CH₃ | H | 3.483 min |
| | | | | | | | m/z = 391.7 [M + H] |
| 31 | F | 3-F | 4-F | CH₃ | CH₃ | H | 3.539 min |
| | | | | | | | m/z = 391.7 [M + H] |
| 32 | Cl | 6-F | H | CH₃ | CH₃ | H | 3.062 min |
| | | | | | | | m/z = 388.8 [M+] |
| 33 | F | 4-CF₃ | H | CH₃ | CH₃ | H | 3.746 min |
| | | | | | | | m/z = 423.7 [M + H] |
| 34 | I | H | H | CH₃ | CH₃ | H | 3.235 min |
| | | | | | | | m/z = 462.6 [M + H] |
| 35 | Cl | 4-NO₂ | 5-Cl | CH₃ | CH₃ | H | 3.638 min |
| | | | | | | | m/z = 452.0 [M + 2] |
| 36 | Cl | 6-F | H | CH₃ | CH₃ | H | 2.926 min |
| | | | | | | | m/z = 388.7 [M] |
| 37 | Cl | 6-CH₃ | 5-Cl | CH₃ | CH₃ | H | 3.134 min |
| | | | | | | | m/z = 421.5 [M + H] |
| 38 | Cl | 5-Cl | H | CH₃ | CH₃ | H | 4.006 min |
| | | | | | | | m/z = 405.8 [M + H] |
| 39 | C₆H₅ | H | H | CH₃ | CH₃ | H | 4.033 min |
| | | | | | | | m/z = 413.9 [M + H] |

Except for the compounds marked ^{*)} and ^{**)}, an isomer mixture was obtained in each case, which was not separated.
^{*)} (S,S)-I (main isomer 1)
^{**)} (R,S)-I (minor isomer 2)
mp. melting point
RT retention time, HPLC

### Use Examples

The herbicidal activity of the compounds of the formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this has been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments belonged to the following species:

| Bayer Code | Scientific name | Common name |
|---|---|---|
| AMARE | Amaranthus retoflexus | redroot pigweed |
| ALOMY | Alopecurus myosuroides | black grass |
| APESV | Apera spica-venti | windgrass |
| CHEAL | Chenopodium album | common lambsquarters |
| ECHCG | Echinochloa crus-galli | barnyard grass |
| SETFA | Setaria faberi | giant foxtail |
| SETVI | Setaria viridis | green foxtail |

The compounds according to the invention, applied by the pre-emergence method, showed very good herbicidal activity.

At an application rate of 1.0 kg/ha, the compound of Example 1 (main isomer 1) applied by the pre-emergence method, showed very good herbicidal activity against AMARE, ALOMY, APESV, ECHCG and SETFA.

At an application rate of 0.5 kg/ha, the compound of Example 1 (minor isomer 2), applied by the pre-emergence method, showed very good herbicidal activity against ALOMY, APESV and ECHCG.

The compounds according to the invention, applied by the post-emergence method, showed very good herbicidal activity.

At an application rate of 1.0 kg/ha, the compound of Example 1 (main isomer 1) applied by the post-emergence method, showed very good herbicidal activity against AMARE, CHEAL, ECHCG and SETVI.

At an application rate of 0.5 kg/ha, the compound of Example 1 (minor isomer 2), applied by the post-emergence method, showed very good herbicidal activity against ALOMY, APESV and ECHCG.

1-16. (canceled)

17. A piperazine compound of the formula I 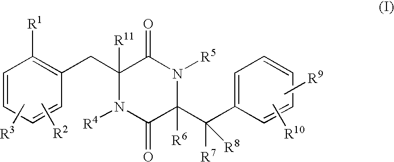
in which
R¹ is selected from the group consisting of halogen, cyano, nitro, Z—C(═O)—R¹², phenyl and a 5- or 6-membered heterocyclic radical which has 1, 2, 3 or 4 heteroatoms selected from the group consisting of O, N and S as ring atoms, where phenyl and the heterocyclic radical are unsubstituted or may have 1, 2, 3 or 4 substituents R^{1a} independently of one another selected from the group consisting of halogen, CN, NO₂, C₁-C₄-alkyl, C₁-C₄-haloalkyl, C₁-C₄-alkoxy and C₁-C₄-haloalkoxy, and in which
Z is a covalent bond or a CH₂ group;
R¹² is hydrogen, C₁-C₆-alkyl, C₃-C₆-cycloalkyl, C₂-C₆-alkenyl, C₅-C₆-cycloalkenyl, C₂-C₆-alkynyl, hydroxyl, C₁-C₆-alkoxy, C₃-C₆-alkenyloxy, C₃-C₆-alkynyloxy, amino, C₁-C₆-alkylamino, (di-(C₁-C₆)-alkyl)amino, C₁-C₆-alkoxyamino, C₁-C₆-alkylsulfonylamino, C₁-C₆-alkylaminosulfonylamino, (di-(C₁-C₆)-alkylamino)sulfonylamino, C₃-C₆-alkenylamino, C₃-C₆-alkynylamino, N—(C₂-C₆-alkenyl)-N—(C₁-C₆-alkyl)-amino, N—(C₂-C₆-alkynyl)-N—(C₁-C₆-alkyl)-amino, N—(C₁-C₆-alkoxy)-N—(C₁-C₆-alkyl)-amino, N—(C₂-C₆-alkenyl)-N—(C₁-C₆-alkoxy)-amino, N—(C₂-C₆-alkynyl)-N—(C₁-C₆-alkoxy)-amino, phenyl, phenoxy or wherein the alkyl moieties in the radicals listed under R¹² may be partially or fully halogenated and the phenyl moieties in the radicals listed under R¹² may carry 1, 2, 3 or 4 substituents R^{12a} selected from the group consisting of halogen, CN, NO₂, C₁-C₄-alkyl, C₁-C₄-haloalkyl, C₁-C₄-alkoxy and C₁-C₄-haloalkoxy;
R² is hydrogen, halogen, nitro, cyano, C₁-C₄-alkyl, C₁-C₄-haloalkyl, C₂-C₄-alkenyl, C₁-C₄-alkoxy, C₁-C₄-haloalkoxy, benzyl or a group S(O)ₙR²¹ in which R²¹ is C₁-C₄-alkyl or C₁-C₄-haloalkyl and n is 0, 1 or 2;
R³ is hydrogen or halogen;
R⁴ is C₁-C₄-alkyl, C₃-C₄-alkenyl or C₃-C₄-alkynyl;
R⁵ is hydrogen, C₁-C₄-alkyl, C₃-C₄-alkenyl, C₃-C₄-alkynyl or a group C(═O)R⁵¹ in which R⁵¹ is hydrogen, C₁-C₄-alkyl, C₁-C₄-haloalkyl, C₁-C₄-alkoxy or C₁-C₄-haloalkoxy;
R⁶ is C₁-C₄-alkyl, C₁-C₄-hydroxyalkyl or C₁-C₄-haloalkyl;
R⁷, R⁸ independently of one another are hydrogen, OH, C₁-C₄-alkoxy, C₁-C₄-haloalkyloxy, C₁-C₄-alkyl or C₁-C₄-haloalkyl;
R⁹, R¹⁰ independently of one another are selected from the group consisting of hydrogen, halogen, CN, NO₂, C₁-C₄-alkyl, C₁-C₄-haloalkyl, C₂-C₄-alkenyl, C₁-C₄-alkoxy and C₁-C₄-haloalkoxy; and
R¹¹ is hydrogen or C₁-C₄-alkyl;
or an agriculturally useful salt of this compound.

18. The piperazine compound according to claim 17 in which R¹ is cyano, nitro or a 5- or 6-membered heteroaromatic radical which has either 1, 2 or 3 nitrogen atoms or 1 oxygen or 1 sulfur atom and, if appropriate, 1 or 2 nitrogen atoms as ring members and which is unsubstituted or may have 1 or 2 substituents selected from R^{1a}.

19. The piperazine compound according to claim 17 in which R¹ is halogen.

20. The piperazine compound according to claim 17 in which R⁴ is

21. The piperazine compound according to claim 17 in which R⁵ is hydrogen, methyl or ethyl.

22. The piperazine compound according to claim 17 in which R⁵ is C(═O)R⁵¹ in which R⁵¹ is hydrogen, C₁-C₄-alkyl or C₁-C₄-haloalkyl.

23. The piperazine compound according to claim 17 in which R⁶ is methyl or ethyl.

24. The piperazine compound according to claim 17 in which R⁷ and R⁸ are hydrogen.

25. The piperazine compound according to claim 17 in which R¹⁰ is hydrogen.

26. The piperazine compound according to claim 17 in which R¹¹ is hydrogen.

27. The piperazine compound according to claim 17 of the general formula Ia in which R¹, R², R³, R⁴, R⁵, R⁶ and R⁹ have one of the meanings given in claim 17: 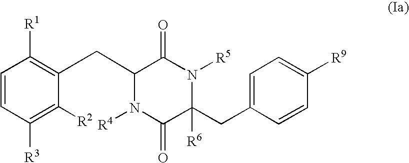
or an agriculturally useful salt of this compound.

28. The piperazine compound according to claim 27 in which
R¹ is cyano or nitro;
R² is hydrogen, fluorine, chlorine, C₁-C₂-alkyl, ethenyl or C₁-C₂-alkoxy;
R³ is fluorine or hydrogen;
R³ is methyl;
R⁵ is hydrogen, methyl or ethyl;
R⁶ is methyl or ethyl; and
R⁹ is hydrogen or halogen.

29. The piperazine compound according to claim 17, selected from the group consisting of:
2-[5-benzyl-1,4,5-trimethyl-3,6-dioxopiperazin-2-ylmethyl]benzonitrile,
2-[5-benzyl-1,4,5-trimethyl-3,6-dioxopiperazin-2-ylmethyl]-3-fluorobenzonitrile,
2-[5-benzyl-1,4,5-trimethyl-3,6-dioxopiperazin-2-ylmethyl]-3-methoxybenzonitrile,
2-[5-benzyl-1,4,5-trimethyl-3,6-dioxopiperazin-2-ylmethyl]-3,4-difluorobenzonitrile,
2-[5-benzyl-1,4,5-trimethyl-3,6-dioxopiperazin-2-ylmethyl]-3-methylbenzonitrile,
2-[5-benzyl-1,4,5-trimethyl-3,6-dioxopiperazin-2-ylmethyl]-3-ethenylbenzonitrile,
2-[5-benzyl-1,5-dimethyl-3,6-dioxopiperazin-2-ylmethyl]benzonitrile,
2-[5-benzyl-1,5-dimethyl-3,6-dioxopiperazin-2-ylmethyl]-3-fluorobenzonitrile,
2-[5-benzyl-1,5-dimethyl-3,6-dioxopiperazin-2-ylmethyl]-3-methoxybenzonitrile,
2-[5-benzyl-1,5-dimethyl-3,6-dioxopiperazin-2-ylmethyl]-3,4-difluorobenzonitrile,
2-[5-benzyl-1,5-dimethyl-3,6-dioxopiperazin-2-ylmethyl]-3-methylbenzonitrile,
2-[5-benzyl-1,5-dimethyl-3,6-dioxopiperazin-2-ylmethyl]-3-ethenylbenzonitrile,
2-[5-benzyl-5-ethyl-1,4-dimethyl-3,6-dioxopiperazin-2-ylmethyl]benzonitrile,
2-[5-benzyl-5-ethyl-1,4-dimethyl-3,6-dioxopiperazin-2-ylmethyl]-3-fluorobenzonitrile,
2-[5-benzyl-5-ethyl-1,4-dimethyl-3,6-dioxopiperazin-2-ylmethyl]-3-methoxybenzonitrile,
2-[5-benzyl-5-ethyl-1,4-dimethyl-3,6-dioxopiperazin-2-ylmethyl]-3,4-difluorobenzonitrile,
2-[5-benzyl-5-ethyl-1,4-dimethyl-3,6-dioxopiperazin-2-ylmethyl]-3-methylbenzonitrile,
2-[5-benzyl-5-ethyl-1,4-dimethyl-3,6-dioxopiperazin-2-ylmethyl]-3-ethenylbenzonitrile,
2-[5-benzyl-5-ethyl-1-methyl-3,6-dioxopiperazin-2-ylmethyl]benzonitrile,
2-[5-benzyl-5-ethyl-1-methyl-3,6-dioxopiperazin-2-ylmethyl]-3-fluorobenzonitrile,
2-[5-benzyl-5-ethyl-1-methyl-3,6-dioxopiperazin-2-ylmethyl]-3-methoxybenzonitrile,
2-[5-benzyl-5-ethyl-1-methyl-3,6-dioxopiperazin-2-ylmethyl]-3,4-difluorobenzonitrile,
2-[5-benzyl-5-ethyl-1-methyl-3,6-dioxopiperazin-2-ylmethyl]-3-methylbenzonitrile,
2-[5-benzyl-5-ethyl-1-methyl-3,6-dioxopiperazin-2-ylmethyl]-3-ethenylbenzonitrile,
2-[5-(4-fluorobenzyl)-1,4,5-trimethyl-3,6-dioxopiperazin-2-ylmethyl]benzonitrile,
2-[5-(4-fluorobenzyl)-1,4,5-trimethyl-3,6-dioxopiperazin-2-ylmethyl]-3-fluorobenzonitrile,
2-[5-(4-fluorobenzyl)-1,4,5-trimethyl-3,6-dioxopiperazin-2-ylmethyl]-3-methoxybenzonitrile,
2-[5-(4-fluorobenzyl)-1,4,5-trimethyl-3,6-dioxopiperazin-2-ylmethyl]-3,4-difluorobenzonitrile,
2-[5-(4-fluorobenzyl)-1,4,5-trimethyl-3,6-dioxopiperazin-2-ylmethyl]-3-methylbenzonitrile,
2-[5-(4-fluorobenzyl)-1,4,5-trimethyl-3,6-dioxopiperazin-2-ylmethyl]-3-ethenylbenzonitrile,
2-[5-(4-fluorobenzyl)-1,5-dimethyl-3,6-dioxopiperazin-2-ylmethyl]benzonitrile,
2-[5-(4-fluorobenzyl)-1,5-dimethyl-3,6-dioxopiperazin-2-ylmethyl]-3-fluorobenzonitrile,
2-[5-(4-fluorobenzyl)-1,5-dimethyl-3,6-dioxopiperazin-2-ylmethyl]-3-methoxybenzonitrile,
2-[5-(4-fluorobenzyl)-1,5-dimethyl-3,6-dioxopiperazin-2-ylmethyl]-3,4-difluorobenzonitrile,
2-[5-(4-fluorobenzyl)-1,5-dimethyl-3,6-dioxopiperazin-2-ylmethyl]-3-methylbenzonitrile,
2-[5-(4-fluorobenzyl)-1,5-dimethyl-3,6-dioxopiperazin-2-ylmethyl]-3-ethenylbenzonitrile,
2-[5-(4-fluorobenzyl)-5-ethyl-1,4-dimethyl-3,6-dioxopiperazin-2-ylmethyl]-benzonitrile,
2-[5-(4-fluorobenzyl)-5-ethyl-1,4-dimethyl-3,6-dioxopiperazin-2-ylmethyl]-3-fluorobenzonitrile,
2-[5-(4-fluorobenzyl)-5-ethyl-1,4-dimethyl-3,6-dioxopiperazin-2-ylmethyl]-3-methoxybenzonitrile,
2-[5-(4-fluorobenzyl)-5-ethyl-1,4-dimethyl-3,6-dioxopiperazin-2-ylmethyl]-3,4-difluorobenzonitrile,
2-[5-(4-fluorobenzyl)-5-ethyl-1,4-dimethyl-3,6-dioxopiperazin-2-ylmethyl]-3-methylbenzonitrile,
2-[5-(4-fluorobenzyl)-5-ethyl-1,4-dimethyl-3,6-dioxopiperazin-2-ylmethyl]-3-ethenylbenzonitrile,
2-[5-(4-fluorobenzyl)-5-ethyl-1-methyl-3,6-dioxopiperazin-2-ylmethyl]benzonitrile,
2-[5-(4-fluorobenzyl)-5-ethyl-1-methyl-3,6-dioxopiperazin-2-ylmethyl]-3-fluorobenzonitrile,
2-[5-(4-fluorobenzyl)-5-ethyl-1-methyl-3,6-dioxopiperazin-2-ylmethyl]-3-methoxybenzonitrile,
2-[5-(4-fluorobenzyl)-5-ethyl-1-methyl-3,6-dioxopiperazin-2-ylmethyl]-3,4-difluorobenzonitrile,
2-[5-(4-fluorobenzyl)-5-ethyl-1-methyl-3,6-dioxopiperazin-2-ylmethyl]-3-methylbenzonitrile,
2-[5-(4-fluorobenzyl)-5-ethyl-1-methyl-3,6-dioxopiperazin-2-ylmethyl]-3-ethenylbenzonitrile,
3-benzyl-6-(2-nitrobenzyl)-1,3,4-trimethylpiperazine-2,5-dione,
3-benzyl-6-(2-fluoro-6-nitrobenzyl)-1,3,4-trimethylpiperazine-2,5-dione,
3-benzyl-6-(2,3-difluoro-6-nitrobenzyl)-1,3,4-trimethylpiperazine-2,5-dione,
3-benzyl-6-(2-methoxy-6-nitrobenzyl)-1,3,4-trimethylpiperazine-2,5-dione,
3-benzyl-6-(2-methyl-6-nitrobenzyl)-1,3,4-trimethylpiperazine-2,5-dione,
3-benzyl-6-(2-ethenyl-6-nitrobenzyl)-1,3,4-trimethylpiperazine-2,5-dione,
3-benzyl-6-(2-nitrobenzyl)-1,3-dimethylpiperazine-2,5-dione,
3-benzyl-6-(2-fluoro-6-nitrobenzyl)-1,3-dimethylpiperazine-2,5-dione,
3-benzyl-6-(2,3-difluoro-6-nitrobenzyl)-1,3-dimethylpiperazine-2,5-dione,
3-benzyl-6-(2-methoxy-6-nitrobenzyl)-1,3-dimethylpiperazine-2,5-dione,
3-benzyl-6-(2-methyl-6-nitrobenzyl)-1,3-dimethylpiperazine-2,5-dione,
3-benzyl-6-(2-ethenyl-6-nitrobenzyl)-1,3-dimethylpiperazine-2,5-dione,
3-benzyl-6-(2-nitrobenzyl)-3-ethyl-1,4-dimethylpiperazine-2,5-dione,
3-benzyl-6-(2-fluoro-6-nitrobenzyl)-3-ethyl-1,4-dimethylpiperazine-2,5-dione,
3-benzyl-6-(2,3-difluoro-6-nitrobenzyl)-3-ethyl-1,4-dimethylpiperazine-2,5-dione,
3-benzyl-6-(2-methoxy-6-nitrobenzyl)-3-ethyl-1,4-dimethylpiperazine-2,5-dione,
3-benzyl-6-(2-methyl-6-nitrobenzyl)-3-ethyl-1,4-dimethylpiperazine-2,5-dione,
3-benzyl-6-(2-ethenyl-6-nitrobenzyl)-3-ethyl-1,4-dimethylpiperazine-2,5-dione,
3-benzyl-6-(2-nitrobenzyl)-3-ethyl-1-methylpiperazine-2,5-dione,
3-benzyl-6-(2-fluoro-6-nitrobenzyl)-3-ethyl-1-methylpiperazine-2,5-dione,
3-benzyl-6-(2,3-difluoro-6-nitrobenzyl)-3-ethyl-1-methylpiperazine-2,5-dione,
3-benzyl-6-(2-methoxy-6-nitrobenzyl)-3-ethyl-1-methylpiperazine-2,5-dione,
3-benzyl-6-(2-methyl-6-nitrobenzyl)-3-ethyl-1-methylpiperazine-2,5-dione,
3-benzyl-6-(2-ethenyl-6-nitrobenzyl)-3-ethyl-1-methylpiperazine-2,5-dione,
3-(4-fluorobenzyl)-6-(2-nitrobenzyl)-1,3,4-trimethylpiperazine-2,5-dione,
3-(4-fluorobenzyl)-6-(2-fluoro-6-nitrobenzyl)-1,3,4-trimethylpiperazine-2,5-dione,
3-(4-fluorobenzyl)-6-(2,3-difluoro-6-nitrobenzyl)-1,3,4-trimethylpiperazine-2,5-dione,
3-(4-fluorobenzyl)-6-(2-methoxy-6-nitrobenzyl)-1,3,4-trimethylpiperazine-2,5-dione,
3-(4-fluorobenzyl)-6-(2-methyl-6-nitrobenzyl)-1,3,4-trimethylpiperazine-2,5-dione,
3-(4-fluorobenzyl)-6-(2-ethenyl-6-nitrobenzyl)-1,3,4-trimethylpiperazine-2,5-dione,
3-(4-fluorobenzyl)-6-(2-nitrobenzyl)-1,3-dimethylpiperazine-2,5-dione,
3-(4-fluorobenzyl)-6-(2-fluoro-6-nitrobenzyl)-1,3-dimethylpiperazine-2,5-dione,
3-(4-fluorobenzyl)-6-(2,3-difluoro-6-nitrobenzyl)-1,3-dimethylpiperazine-2,5-dione,
3-(4-fluorobenzyl)-6-(2-methoxy-6-nitrobenzyl)-1,3-dimethylpiperazine-2,5-dione,
3-(4-fluorobenzyl)-6-(2-methyl-6-nitrobenzyl)-1,3-dimethylpiperazine-2,5-dione,
3-(4-fluorobenzyl)-6-(2-ethenyl-6-nitrobenzyl)-1,3-dimethylpiperazine-2,5-dione,
3-(4-fluorobenzyl)-6-(2-nitrobenzyl)-3-ethyl-1,4-dimethylpiperazine-2,5-dione,
3-(4-fluorobenzyl)-6-(2-fluoro-6-nitrobenzyl)-3-ethyl-1,4-dimethylpiperazine-2,5-dione,
3-(4-fluorobenzyl)-6-(2,3-difluoro-6-nitrobenzyl)-3-ethyl-1,4-dimethylpiperazine-2,5-dione,
3-(4-fluorobenzyl)-6-(2-methoxy-6-nitrobenzyl)-3-ethyl-1,4-dimethylpiperazine-2,5-dione,
3-(4-fluorobenzyl)-6-(2-methyl-6-nitrobenzyl)-3-ethyl-1,4-dimethylpiperazine-2,5-dione,
3-(4-fluorobenzyl)-6-(2-ethenyl-6-nitrobenzyl)-3-ethyl-1,4-dimethylpiperazine-2,5-dione,
3-(4-fluorobenzyl)-6-(2-nitrobenzyl)-3-ethyl-1-methylpiperazine-2,5-dione,
3-(4-fluorobenzyl)-6-(2-fluoro-6-nitrobenzyl)-3-ethyl-1-methylpiperazine-2,5-dione,
3-(4-fluorobenzyl)-6-(2,3-difluoro-6-nitrobenzyl)-3-ethyl-1-methylpiperazine-2,5-dione,
3-(4-fluorobenzyl)-6-(2-methoxy-6-nitrobenzyl)-3-ethyl-1-methylpiperazine-2,5-dione,
3-(4-fluorobenzyl)-6-(2-methyl-6-nitrobenzyl)-3-ethyl-1-methylpiperazine-2,5-dione, and
3-(4-fluorobenzyl)-6-(2-ethenyl-6-nitrobenzyl)-3-ethyl-1-methylpiperazine-2,5-dione.

30. A composition comprising a herbicidally effective amount of at least one piperazine compound of the formula I, or an agriculturally useful salt thereof, according to claim 17 and auxiliaries customary for formulating crop protection agents.

31. The composition of claim 30, wherein the compound has the general formula Ia: 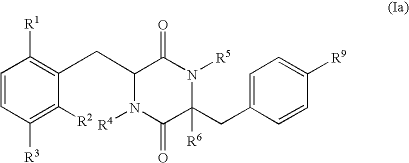
or an agriculturally useful salt thereof.

32. A method for controlling unwanted vegetation wherein a herbicidally effective amount of at least one piperazine compound of the formula I or Ia or an agriculturally useful salt of I according claim 17 is allowed to act on plants, their seed and/or their habitat.

33. The method of claim 32 wherein the compound has the general formula Ia: 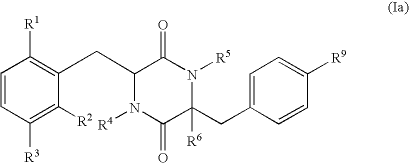
or an agriculturally useful salt thereof.

34. The method of claim 32, wherein the compound is selected from the group consisting of:
2-[5-benzyl-1,4,5-trimethyl-3,6-dioxopiperazin-2-ylmethyl]benzonitrile,
2-[5-benzyl-1,4,5-trimethyl-3,6-dioxopiperazin-2-ylmethyl]-3-fluorobenzonitrile,
2-[5-benzyl-1,4,5-trimethyl-3,6-dioxopiperazin-2-ylmethyl]-3-methoxybenzonitrile,
2-[5-benzyl-1,4,5-trimethyl-3,6-dioxopiperazin-2-ylmethyl]-3,4-difluorobenzonitrile,
2-[5-benzyl-1,4,5-trimethyl-3,6-dioxopiperazin-2-ylmethyl]-3-methylbenzonitrile,
2-[5-benzyl-1,4,5-trimethyl-3,6-dioxopiperazin-2-ylmethyl]-3-ethenylbenzonitrile,
2-[5-benzyl-1,5-dimethyl-3,6-dioxopiperazin-2-ylmethyl]benzonitrile,
2-[5-benzyl-1,5-dimethyl-3,6-dioxopiperazin-2-ylmethyl]-3-fluorobenzonitrile,
2-[5-benzyl-1,5-dimethyl-3,6-dioxopiperazin-2-ylmethyl]-3-methoxybenzonitrile,
2-[5-benzyl-1,5-dimethyl-3,6-dioxopiperazin-2-ylmethyl]-3,4-difluorobenzonitrile,
2-[5-benzyl-1,5-dimethyl-3,6-dioxopiperazin-2-ylmethyl]-3-methylbenzonitrile,
2-[5-benzyl-1,5-dimethyl-3,6-dioxopiperazin-2-ylmethyl]-3-ethenylbenzonitrile,
2-[5-benzyl-5-ethyl-1,4-dimethyl-3,6-dioxopiperazin-2-ylmethyl]benzonitrile,
2-[5-benzyl-5-ethyl-1,4-dimethyl-3,6-dioxopiperazin-2-ylmethyl]-3-fluorobenzonitrile,
2-[5-benzyl-5-ethyl-1,4-dimethyl-3,6-dioxopiperazin-2-ylmethyl]-3-methoxybenzonitrile,
2-[5-benzyl-5-ethyl-1,4-dimethyl-3,6-dioxopiperazin-2-ylmethyl]-3,4-difluorobenzonitrile,
2-[5-benzyl-5-ethyl-1,4-dimethyl-3,6-dioxopiperazin-2-ylmethyl]-3-methylbenzonitrile,
2-[5-benzyl-5-ethyl-1,4-dimethyl-3,6-dioxopiperazin-2-ylmethyl]-3-ethenylbenzonitrile,
2-[5-benzyl-5-ethyl-1-methyl-3,6-dioxopiperazin-2-ylmethyl]benzonitrile,
2-[5-benzyl-5-ethyl-1-methyl-3,6-dioxopiperazin-2-ylmethyl]-3-fluorobenzonitrile,
2-[5-benzyl-5-ethyl-1-methyl-3,6-dioxopiperazin-2-ylmethyl]-3-methoxybenzonitrile,
2-[5-benzyl-5-ethyl-1-methyl-3,6-dioxopiperazin-2-ylmethyl]-3,4-difluorobenzonitrile,
2-[5-benzyl-5-ethyl-1-methyl-3,6-dioxopiperazin-2-ylmethyl]-3-methylbenzonitrile,
2-[5-benzyl-5-ethyl-1-methyl-3,6-dioxopiperazin-2-ylmethyl]-3-ethenylbenzonitrile,
2-[5-(4-fluorobenzyl)-1,4,5-trimethyl-3,6-dioxopiperazin-2-ylmethyl]benzonitrile,
2-[5-(4-fluorobenzyl)-1,4,5-trimethyl-3,6-dioxopiperazin-2-ylmethyl]-3-fluorobenzonitrile,
2-[5-(4-fluorobenzyl)-1,4,5-trimethyl-3,6-dioxopiperazin-2-ylmethyl]-3-methoxybenzonitrile,
2-[5-(4-fluorobenzyl)-1,4,5-trimethyl-3,6-dioxopiperazin-2-ylmethyl]-3,4-difluorobenzonitrile,
2-[5-(4-fluorobenzyl)-1,4,5-trimethyl-3,6-dioxopiperazin-2-ylmethyl]-3-methylbenzonitrile,
2-[5-(4-fluorobenzyl)-1,4,5-trimethyl-3,6-dioxopiperazin-2-ylmethyl]-3-ethenylbenzonitrile,
2-[5-(4-fluorobenzyl)-1,5-dimethyl-3,6-dioxopiperazin-2-ylmethyl]benzonitrile,
2-[5-(4-fluorobenzyl)-1,5-dimethyl-3,6-dioxopiperazin-2-ylmethyl]-3-fluorobenzonitrile,
2-[5-(4-fluorobenzyl)-1,5-dimethyl-3,6-dioxopiperazin-2-ylmethyl]-3-methoxybenzonitrile,
2-[5-(4-fluorobenzyl)-1,5-dimethyl-3,6-dioxopiperazin-2-ylmethyl]-3,4-difluorobenzonitrile,
2-[5-(4-fluorobenzyl)-1,5-dimethyl-3,6-dioxopiperazin-2-ylmethyl]-3-methylbenzonitrile,
2-[5-(4-fluorobenzyl)-1,5-dimethyl-3,6-dioxopiperazin-2-ylmethyl]-3-ethenylbenzonitrile,
2-[5-(4-fluorobenzyl)-5-ethyl-1,4-dimethyl-3,6-dioxopiperazin-2-ylmethyl]-benzonitrile,
2-[5-(4-fluorobenzyl)-5-ethyl-1,4-dimethyl-3,6-dioxopiperazin-2-ylmethyl]-3-fluorobenzonitrile,
2-[5-(4-fluorobenzyl)-5-ethyl-1,4-dimethyl-3,6-dioxopiperazin-2-ylmethyl]-3-methoxybenzonitrile,
2-[5-(4-fluorobenzyl)-5-ethyl-1,4-dimethyl-3,6-dioxopiperazin-2-ylmethyl]-3,4-difluorobenzonitrile,
2-[5-(4-fluorobenzyl)-5-ethyl-1,4-dimethyl-3,6-dioxopiperazin-2-ylmethyl]-3-methylbenzonitrile,
2-[5-(4-fluorobenzyl)-5-ethyl-1,4-dimethyl-3,6-dioxopiperazin-2-ylmethyl]-3-ethenylbenzonitrile,
2-[5-(4-fluorobenzyl)-5-ethyl-1-methyl-3,6-dioxopiperazin-2-ylmethyl]benzonitrile,
2-[5-(4-fluorobenzyl)-5-ethyl-1-methyl-3,6-dioxopiperazin-2-ylmethyl]-3-fluorobenzonitrile,
2-[5-(4-fluorobenzyl)-5-ethyl-1-methyl-3,6-dioxopiperazin-2-ylmethyl]-3-methoxybenzonitrile,
2-[5-(4-fluorobenzyl)-5-ethyl-1-methyl-3,6-dioxopiperazin-2-ylmethyl]-3,4-difluorobenzonitrile,
2-[5-(4-fluorobenzyl)-5-ethyl-1-methyl-3,6-dioxopiperazin-2-ylmethyl]-3-methylbenzonitrile,
2-[5-(4-fluorobenzyl)-5-ethyl-1-methyl-3,6-dioxopiperazin-2-ylmethyl]-3-ethenylbenzonitrile,
3-benzyl-6-(2-nitrobenzyl)-1,3,4-trimethylpiperazine-2,5-dione,
3-benzyl-6-(2-fluoro-6-nitrobenzyl)-1,3,4-trimethylpiperazine-2,5-dione,
3-benzyl-6-(2,3-difluoro-6-nitrobenzyl)-1,3,4-trimethylpiperazine-2,5-dione,
3-benzyl-6-(2-methoxy-6-nitrobenzyl)-1,3,4-trimethylpiperazine-2,5-dione,
3-benzyl-6-(2-methyl-6-nitrobenzyl)-1,3,4-trimethylpiperazine-2,5-dione,
3-benzyl-6-(2-ethenyl-6-nitrobenzyl)-1,3,4-trimethylpiperazine-2,5-dione,
3-benzyl-6-(2-nitrobenzyl)-1,3-dimethylpiperazine-2,5-dione,
3-benzyl-6-(2-fluoro-6-nitrobenzyl)-1,3-dimethylpiperazine-2,5-dione,
3-benzyl-6-(2,3-difluoro-6-nitrobenzyl)-1,3-dimethylpiperazine-2,5-dione,
3-benzyl-6-(2-methoxy-6-nitrobenzyl)-1,3-dimethylpiperazine-2,5-dione,
3-benzyl-6-(2-methyl-6-nitrobenzyl)-1,3-dimethylpiperazine-2,5-dione,
3-benzyl-6-(2-ethenyl-6-nitrobenzyl)-1,3-dimethylpiperazine-2,5-dione,
3-benzyl-6-(2-nitrobenzyl)-3-ethyl-1,4-dimethylpiperazine-2,5-dione,
3-benzyl-6-(2-fluoro-6-nitrobenzyl)-3-ethyl-1,4-dimethylpiperazine-2,5-dione,
3-benzyl-6-(2,3-difluoro-6-nitrobenzyl)-3-ethyl-1,4-dimethylpiperazine-2,5-dione,
3-benzyl-6-(2-methoxy-6-nitrobenzyl)-3-ethyl-1,4-dimethylpiperazine-2,5-dione,
3-benzyl-6-(2-methyl-6-nitrobenzyl)-3-ethyl-1,4-dimethylpiperazine-2,5-dione,
3-benzyl-6-(2-ethenyl-6-nitrobenzyl)-3-ethyl-1,4-dimethylpiperazine-2,5-dione,
3-benzyl-6-(2-nitrobenzyl)-3-ethyl-1-methylpiperazine-2,5-dione,
3-benzyl-6-(2-fluoro-6-nitrobenzyl)-3-ethyl-1-methylpiperazine-2,5-dione,
3-benzyl-6-(2,3-difluoro-6-nitrobenzyl)-3-ethyl-1-methylpiperazine-2,5-dione,
3-benzyl-6-(2-methoxy-6-nitrobenzyl)-3-ethyl-1-methylpiperazine-2,5-dione,
3-benzyl-6-(2-methyl-6-nitrobenzyl)-3-ethyl-1-methylpiperazine-2,5-dione,
3-benzyl-6-(2-ethenyl-6-nitrobenzyl)-3-ethyl-1-methylpiperazine-2,5-dione,
3-(4-fluorobenzyl)-6-(2-nitrobenzyl)-1,3,4-trimethylpiperazine-2,5-dione,
3-(4-fluorobenzyl)-6-(2-fluoro-6-nitrobenzyl)-1,3,4-trimethylpiperazine-2,5-dione,
3-(4-fluorobenzyl)-6-(2,3-difluoro-6-nitrobenzyl)-1,3,4-trimethylpiperazine-2,5-dione,
3-(4-fluorobenzyl)-6-(2-methoxy-6-nitrobenzyl)-1,3,4-trimethylpiperazine-2,5-dione,
3-(4-fluorobenzyl)-6-(2-methyl-6-nitrobenzyl)-1,3,4-trimethylpiperazine-2,5-dione,
3-(4-fluorobenzyl)-6-(2-ethenyl-6-nitrobenzyl)-1,3,4-trimethylpiperazine-2,5-dione,
3-(4-fluorobenzyl)-6-(2-nitrobenzyl)-1,3-dimethylpiperazine-2,5-dione,
3-(4-fluorobenzyl)-6-(2-fluoro-6-nitrobenzyl)-1,3-dimethylpiperazine-2,5-dione,
3-(4-fluorobenzyl)-6-(2,3-difluoro-6-nitrobenzyl)-1,3-dimethylpiperazine-2,5-dione,
3-(4-fluorobenzyl)-6-(2-methoxy-6-nitrobenzyl)-1,3-dimethylpiperazine-2,5-dione,
3-(4-fluorobenzyl)-6-(2-methyl-6-nitrobenzyl)-1,3-dimethylpiperazine-2,5-dione,
3-(4-fluorobenzyl)-6-(2-ethenyl-6-nitrobenzyl)-1,3-dimethylpiperazine-2,5-dione,
3-(4-fluorobenzyl)-6-(2-nitrobenzyl)-3-ethyl-1,4-dimethylpiperazine-2,5-dione,
3-(4-fluorobenzyl)-6-(2-fluoro-6-nitrobenzyl)-3-ethyl-1,4-dimethylpiperazine-2,5-dione,
3-(4-fluorobenzyl)-6-(2,3-difluoro-6-nitrobenzyl)-3-ethyl-1,4-dimethylpiperazine-2,5-dione,
3-(4-fluorobenzyl)-6-(2-methoxy-6-nitrobenzyl)-3-ethyl-1,4-dimethylpiperazine-2,5-dione,
3-(4-fluorobenzyl)-6-(2-methyl-6-nitrobenzyl)-3-ethyl-1,4-dimethylpiperazine-2,5-dione,
3-(4-fluorobenzyl)-6-(2-ethenyl-6-nitrobenzyl)-3-ethyl-1,4-dimethylpiperazine-2,5-dione,
3-(4-fluorobenzyl)-6-(2-nitrobenzyl)-3-ethyl-1-methylpiperazine-2,5-dione,
3-(4-fluorobenzyl)-6-(2-fluoro-6-nitrobenzyl)-3-ethyl-1-methylpiperazine-2,5-dione,
3-(4-fluorobenzyl)-6-(2,3-difluoro-6-nitrobenzyl)-3-ethyl-1-methylpiperazine-2,5-dione,
3-(4-fluorobenzyl)-6-(2-methoxy-6-nitrobenzyl)-3-ethyl-1-methylpiperazine-2,5-dione,
3-(4-fluorobenzyl)-6-(2-methyl-6-nitrobenzyl)-3-ethyl-1-methylpiperazine-2,5-dione, and
3-(4-fluorobenzyl)-6-(2-ethenyl-6-nitrobenzyl)-3-ethyl-1-methylpiperazine-2,5-dione.

35. The method of claim 32 in which R¹ is cyano, nitro or a 5- or 6-membered heteroaromatic radical which has either 1, 2 or 3 nitrogen atoms or 1 oxygen or 1 sulfur atom and, if appropriate, 1 or 2 nitrogen atoms as ring members and which is unsubstituted or may have 1 or 2 substituents selected from R^{1a}.

36. The method of claim 32 in which R¹ is halogen.
